SEQUENCE LISTING (23 sequences)

SEQ ID NO: 1 - DNA, 30 bp, synthetic construct
  source 1..30: mol_type = other DNA
gcttagccgcttaataacatctaatgcgta

SEQ ID NO: 2 - DNA, 29 bp, synthetic construct
  source 1..29: mol_type = other DNA
cttagccgcttaataacttagctcgcgta

SEQ ID NO: 3 - DNA, 30 bp, synthetic construct
  source 1..30: mol_type = other DNA
gcttagccgcttaataacctcgcttgcgta

SEQ ID NO: 4 - DNA, 15 bp, synthetic construct
  source 1..15: mol_type = other DNA
ttttttttttttttt

SEQ ID NO: 5 - DNA, 15 bp, synthetic construct
  source 1..15: mol_type = other DNA
cccccccccccgggg

SEQ ID NO: 6 - DNA, 15 bp, synthetic construct
  source 1..15: mol_type = other DNA
gaaaaaaagggcccc

SEQ ID NO: 7 - DNA, 15 bp, synthetic construct
  source 1..15: mol_type = other DNA
agggggggaaaaaaa

SEQ ID NO: 8 - DNA, 15 bp, synthetic construct
  source 1..15: mol_type = other DNA
gaaaaaaaaggcccc

SEQ ID NO: 9 - DNA, 15 bp, synthetic construct
  source 1..15: mol_type = other DNA
ccccccccccccggc

SEQ ID NO: 10 - DNA, 15 bp, synthetic construct
  source 1..15: mol_type = other DNA
gaaaaaaaagggccg

SEQ ID NO: 11 - DNA, 15 bp, synthetic construct
  source 1..15: mol_type = other DNA
aggggggggaaaaaa

SEQ ID NO: 12 - DNA, 15 bp, synthetic construct
  source 1..15: mol_type = other DNA
aacaatggccatagg

SEQ ID NO: 13 - DNA, 15 bp, synthetic construct
  source 1..15: mol_type = other DNA
atttcagattgtgtc

SEQ ID NO: 14 - DNA, 15 bp, synthetic construct
  source 1..15: mol_type = other DNA
gttgcgggacaacgg

SEQ ID NO: 15 - DNA, 15 bp, synthetic construct
  source 1..15: mol_type = other DNA
gtccactccggatct

SEQ ID NO: 16 - DNA, 15 bp, synthetic construct
  source 1..15: mol_type = other DNA
ggaggaagtatgttc

SEQ ID NO: 17 - DNA, 15 bp, synthetic construct
  source 1..15: mol_type = other DNA
gattcaatgccgcga

SEQ ID NO: 18 - DNA, 14 bp, synthetic construct
  source 1..14: mol_type = other DNA
gaccgatatctgaa

SEQ ID NO: 19 - DNA, 15 bp, synthetic construct
  source 1..15: mol_type = other DNA
taatccctaagtctt

SEQ ID NO: 20 - DNA, 15 bp, synthetic construct
  source 1..15: mol_type = other DNA
ctcagaatgccatca

SEQ ID NO: 21 - DNA, 15 bp, synthetic construct
  source 1..15: mol_type = other DNA
ttacaaacctttttt

SEQ ID NO: 22 - DNA, 15 bp, synthetic construct
  source 1..15: mol_type = other DNA
taaacactattaagg

SEQ ID NO: 23 - protein, 6 aa, synthetic construct
HHHHHH

(19) United States
(12) Patent Application Publication
(10) Pub. No.: US 2026/0337602 A1
(43) Pub. Date: Oct. 29, 2026

(54) MONITORING POPULATIONS OF CELLS PRODUCING BIOMOLECULES

(71) Applicant: Hoffmann-La Roche Inc., Little Falls, NJ (US)

(72) Inventors: Simon AUSLAENDER, Penzberg (DE); Niels BAUER, Penzberg (DE); Oliver POPP, Penzberg (DE)

(21) Appl. No.: 19/694,827

(22) Filed: Jun. 1, 2026

Related U.S. Application Data
(63) Continuation of application No. PCT/EP2024/084633, filed on Dec. 4, 2024.

(30) Foreign Application Priority Data
Dec. 5, 2023 (EP) 23214270.3

(51) Int. Cl. C12N 15/10; C12Q 1/68; C40B 20/04; C40B 30/00; C40B 40/02; C40B 50/06

(52) U.S. Cl. CPC C12N 15/1082; C12Q 1/68; C40B 20/04; C40B 30/00; C40B 40/02; C40B 50/06

(57) ABSTRACT

The present disclosure relates to analysis of cells obtained by: (a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and (b) subjecting the cells obtained after step (a) to selection for cells having integrated a polynucleotide of interest into their genomic DNA, in order to evaluate/monitor and/or manipulate their diversity.

### CROSS REFERENCE TO RELATED APPLICATIONS

This application is a continuation of International Application No. PCT/EP2024/084633, filed on Dec. 4, 2024, which claims priority to European Patent Application No. EP23214270.3, filed on Dec. 5, 2023, the contents and elements of each of which are herein incorporated by reference for all purposes.

### REFERENCE TO AN ELECTRONIC SEQUENCE LISTING

The content of the electronic sequence listing (256602007201seqlist.xml; Size: 21,188 bytes; and Date of Creation: Jun. 1, 2026) is herein incorporated by reference in its entirety.

### TECHNICAL FIELD

The present disclosure relates to the fields of molecular and cell biology, and cell culture. In particular, the present disclosure relates to monitoring and/or managing the diversity of populations of cells producing biomolecules.

### BACKGROUND

Methods for obtaining monoclonal populations of cells engineered for the production of biomolecules are well known, and are described e.g. in Green and Sambrook, Molecular Cloning: A Laboratory Manual (4th Edition), Cold Spring Harbor Press, 2012 and Nat Methods. (2008); 5 (2): 135-146. Such procedures include limiting dilution from a polyclonal population of engineered cells.

Cells engineered to comprise an integrated polynucleotide of interest produced via distinct integration events can differ considerably in characteristics of relevance to their suitability to be employed for the production of biomolecules of interest, e.g. at the scale for commercial production. For example, the cells produced by different integration events can have different phenotypes relevant to biomolecule production, such as variable growth characteristics, metabolic profiles, optimal culture conditions, productivity, etc.

### SUMMARY

In a first aspect, the present disclosure provides a method for evaluating the diversity of a library of genetically non-identical cells having a polynucleotide of interest integrated into their genomic DNA, comprising analyzing cells obtained by:
(a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
(b) subjecting the cells obtained after step (a) to selection for cells having integrated a polynucleotide of interest into their genomic DNA;
to determine the identity of the identifier sequences integrated into their genomic DNA.

In some embodiments, the method further comprises determining the proportion of cells in the population having integrated a given identifier sequence.

The present disclosure also provides a method for obtaining a library of genetically non-identical cells having a polynucleotide of interest integrated into their genomic DNA, comprising:
(i) analyzing cells obtained by:
   (a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
   (b) subjecting the cells obtained after step (a) to selection for cells having integrated a polynucleotide of interest into their genomic DNA;
to determine the identity of the identifier sequences integrated into their genomic DNA; and
(ii) selecting cells determined in step (i) to have non-identical identifier sequences integrated into their genomic DNA for subsequent expansion.

The present disclosure also provides a method for obtaining a monoclonal population of cells having a polynucleotide of interest integrated into their genomic DNA, comprising:
(i) analyzing cells obtained by:
   (a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
   (b) subjecting the cells obtained after step (a) to selection for cells having integrated a polynucleotide of interest into their genomic DNA;
to determine the identity of the identifier sequences integrated into their genomic DNA; and
(ii) selecting a single cell, or selecting a plurality of cells determined in step (i) to have identical identifier sequences integrated into their genomic DNA, for subsequent expansion.

The present disclosure also provides a method for evaluating the diversity of a population of cells having a polynucleotide of interest integrated into their genomic DNA, comprising analyzing cells obtained by:
(a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
(b) subjecting the cells obtained after step (a) to selection for cells having integrated a polynucleotide of interest into their genomic DNA;
(c) analyzing cells obtained after step (b) to determine the identity of the identifier sequences integrated into their genomic DNA; and
(d) selecting cells determined in step (c) to have non-identical identifier sequences integrated into their genomic DNA for subsequent expansion;
to determine the identity of the identifier sequences integrated into their genomic DNA.

The present disclosure also provides a method for confirming the identity of a population of cells having a polynucleotide of interest integrated into their genomic DNA, comprising analyzing cells obtained by:
(a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
(b) subjecting the cells obtained after step (a) to selection for cells having integrated a polynucleotide of interest into their genomic DNA;
(c) analyzing cells obtained after step (b) to determine the identity of the identifier sequences integrated into their genomic DNA; and
(d) selecting a single cell, or selecting a plurality of cells determined in step (c) to have identical identifier sequences integrated into their genomic DNA, for subsequent expansion;
to determine the identity of the identifier sequences integrated into their genomic DNA.

The present disclosure also provides a method for evaluating the diversity of a library of genetically non-identical cells having a polynucleotide of interest integrated into their genomic DNA, comprising:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA; and
(iii) analyzing cells obtained after step (ii) to determine the identity of the identifier sequences integrated into their genomic DNA.

In some embodiments, the method further comprises determining the proportion of cells in the population obtained after step (ii) having integrated a given identifier sequence.

The present disclosure also provides a method for obtaining a library of genetically non-identical cells having a polynucleotide of interest integrated into their genomic DNA, comprising:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting cells determined in step (iii) to have non-identical identifier sequences integrated into their genomic DNA for subsequent expansion.

The present disclosure also provides a method for obtaining a monoclonal population of cells having a polynucleotide of interest integrated into their genomic DNA, comprising:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion.

The present disclosure also provides a method for evaluating the diversity of a population of cells having a polynucleotide of interest integrated into their genomic DNA, comprising:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion; and
(v) analyzing cells obtained after step (iv) to determine the identity of the identifier sequences integrated into their genomic DNA.

In some embodiments, the method for evaluating the diversity of a population of cells having a polynucleotide of interest integrated into their genomic DNA is a method for monitoring or evaluating the monoclonality of a putative monoclonal population of cells having a polynucleotide of interest integrated into their genomic DNA.

The present disclosure also provides a method for confirming the identity of a population of cells having a polynucleotide of interest integrated into their genomic DNA, comprising:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion; and
(v) analyzing cells obtained after step (iv) to determine the identity of the identifier sequences integrated into their genomic DNA.

In some embodiments, the method for confirming the identity of a population of cells having a polynucleotide of interest integrated into their genomic DNA is a method for confirming the monoclonality of a putative monoclonal population of cells having a polynucleotide of interest integrated into their genomic DNA.

The present disclosure also provides the use of a plurality of vectors comprising nucleotide sequences providing for targeted integration of a polynucleotide of interest comprising an identifier sequence into the genomic DNA of a cell in a method for evaluating the diversity of a library of genetically non-identical cells having a polynucleotide of interest integrated into their genomic DNA, wherein in each vector of the plurality of vectors that comprises a polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a polynucleotide of interest into their genomic DNA; and
(iii) analyzing cells obtained after step (ii) to determine the identity of the identifier sequences integrated into their genomic DNA.

In some embodiments, the method further comprises determining the proportion of cells in the population obtained after step (ii) having integrated a given identifier sequence.

The present disclosure also provides the use of a plurality of vectors comprising nucleotide sequences providing for targeted integration of a polynucleotide of interest comprising an identifier sequence into the genomic DNA of a cell in a method for obtaining a clonal population of cells having a polynucleotide of interest integrated into their genomic DNA, wherein in each vector of the plurality of vectors that comprises a polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting cells determined in step (iii) to have non-identical identifier sequences integrated into their genomic DNA for subsequent expansion.

The present disclosure also provides the use of a plurality of vectors comprising nucleotide sequences providing for targeted integration of a polynucleotide of interest comprising an identifier sequence into the genomic DNA of a cell in a method for obtaining a library of genetically non-identical cells having a polynucleotide of interest integrated into their genomic DNA, wherein in each vector of the plurality of vectors that comprises a polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion.

The present disclosure also provides the use of a plurality of vectors comprising nucleotide sequences providing for targeted integration of a polynucleotide of interest comprising an identifier sequence into the genomic DNA of a cell in a method for evaluating the diversity of a population of cells having a polynucleotide of interest integrated into their genomic DNA, wherein in each vector of the plurality of vectors that comprises a polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion; and
(v) analyzing cells obtained after step (iv) to determine the identity of the identifier sequences integrated into their genomic DNA.

In some embodiments, the method for evaluating the diversity of a population of cells having a polynucleotide of interest integrated into their genomic DNA is a method for monitoring or evaluating the monoclonality of a putative monoclonal population of cells having a polynucleotide of interest integrated into their genomic DNA.

The present disclosure also provides the use of a plurality of vectors comprising nucleotide sequences providing for targeted integration of a polynucleotide of interest comprising an identifier sequence into the genomic DNA of a cell in a method for confirming the identity of a population of cells having a polynucleotide of interest integrated into their genomic DNA, wherein in each vector of the plurality of vectors that comprises a polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion; and
(v) analyzing cells obtained after step (iv) to determine the identity of the identifier sequences integrated into their genomic DNA.

In some embodiments, the method for confirming the identity of a population of cells having a polynucleotide of interest integrated into their genomic DNA is a method for confirming the monoclonality of a putative monoclonal population of cells having a polynucleotide of interest integrated into their genomic DNA.

In some embodiments in accordance with the various aspects of the present disclosure, prior to integration of a polynucleotide of interest, the cells comprise genomic DNA having at least one single-copy landing pad providing for targeted integration of a polynucleotide of interest.

In some embodiments, the polynucleotide of interest comprises a nucleotide sequence encoding one or more polypeptides of interest. In some embodiments, the one or more polypeptides of interest are each independently selected from the group consisting of: an antigen-binding polypeptide, an aptamer, a constituent polypeptide of an antigen-binding polypeptide complex, an antibody/antigen-binding fragment or derivative thereof, a constituent polypeptide of an antibody/antigen-binding fragment or derivative thereof, an Fc fusion polypeptide, an anticoagulant, a blood factor, a bone morphogenetic polypeptide, a decoy receptor for a ligand, a decoy ligand for a receptor, an enzyme, an enzyme co-factor, a growth factor, a hormone, an interferon, an interleukin, a thrombolytic, a transcription factor, an epigenetic modifier, a constituent polypeptide of a site-specific nuclease nucleic acid editing system, a constituent polypeptide of a ribonucleoprotein, a viral polypeptide, or a polypeptide useful for the production of a biomolecule.

### DESCRIPTION

The present disclosure broadly concerns the application of DNA barcoding technology to monitor and/or manage the genetic diversity of cells in culture, in particular cells expressing polypeptides of interest.

The dynamics of producer cell populations play a pivotal role in the consistency and yield of polypeptide production. These dynamics encompass various production-relevant factors such as the growth rates, cell survival capabilities, and productivity of individual cells, as well as the overall clonal composition of the population.

The generation of clonal cell lines has been used to ensure identical genetic makeup and to effectively control the dynamics of producer cells, thus improving consistency, reproducibility and safety of the biomolecules for use in therapeutic applications. However, such cell lines still display a wide array of production-relevant phenotypes necessitating time-consuming and resource-intensive clone screening processes.

The present disclosure applies unique identifier sequences to stable producer cell pools, for example CHO cells. The introduction of unique identifier sequences into polynucleotides for targeted, single-copy integration enables the fate of stable producer pools, and the individual clones thereof, to be monitored, permitting detailed population dynamics of stable producer cell populations to be observed. This is useful for maintaining stable, high-yielding cell lines for therapeutic protein production, and provides for the potentially unlimited labeling and tracking of large populations of cells.

### Polynucleotides of Interest

The present disclosure relates to the production of cells having a polynucleotide of interest integrated into their genomic DNA, through introduction into cells of vectors having nucleotide sequences providing for targeted integration of a polynucleotide of interest.

A ‘polynucleotide’ refers to a polymer chain of a plurality of nucleotide monomers linked by bonds between the monomers, typically phosphodiester bonds (e.g. in the case of polynucleotides formed by naturally-occurring nucleotide monomers). Polynucleotides include oligonucleotides, which generally comprise≤50 nucleotides. A polynucleotide may be single-stranded, or may be double-stranded (i.e. may comprise a duplex formed by hydrogen-bonding between complementary nucleotides). A polynucleotide according to the present disclosure may comprise or consist of double-stranded DNA.

In some embodiments, a polynucleotide according to the present disclosure comprises or consists of DNA. In some embodiments, a polynucleotide is a polydeoxyribonucleotide. In some embodiments, a polynucleotide comprises or consists of double-stranded DNA.

Polynucleotides of the present disclosure are defined herein by reference to constituent nucleotide sequences. It will be appreciated that constituent nucleotide sequences of a polynucleotide of interest according to the present disclosure are provided as subsequences of the complete sequence of the polynucleotide of interest.

Polynucleotides of interest according to the present disclosure comprise an identifier sequence.

As used herein, an ‘identifier sequence’ refers to a nucleotide sequence (i.e. of a polynucleotide of interest) capable of serving as an entity for identification of a polynucleotide of interest comprising the identifier sequence. Polynucleotides are well suited to be used as identifiers, as they are highly polymorphic (and it is therefore possible to produce a very large diversity of unique identifier sequences), and techniques for their detection with high sensitivity and specificity are widely available (e.g. next-generation sequencing technologies). Identifier sequences may also be referred to as ‘barcodes’, and labeling of polynucleotides of interest/vectors with non-identical identifier sequences may be referred to as ‘barcoding’. It will be appreciated that within a given plurality of identifier sequences, each identifier sequence has a unique nucleotide sequence.

In some embodiments, an identifier sequence according to the present disclosure comprises or consists of a DNA polynucleotide (i.e. a polydeoxyribonucleotide). In some embodiments, an identifier sequence comprises or consists of a nucleotide sequence having 5 to 100 nucleotides, e.g. one of 7 to 80, 5 to 50 or 10 to 25 nucleotides.

In some embodiments, within a given plurality of identifier sequences, each identifier sequence comprises one or more (e.g. 2 to 25, 2 to 20, 5 to 15 or 5 to 10) positions that are invariant between the identifier sequences of the plurality. That is, in addition to a sequence of nucleotides that is unique to an individual identifier sequence of the plurality, the identifier sequence may additionally comprise a sequence of nucleotides (e.g. comprising or consisting of 2 to 25, 2 to 20, 5 to 15 or 5 to 10 nucleotides) that is conserved between, and common to, the identifier sequences of the plurality.

Accordingly, in some embodiments, an identifier sequence of a plurality of identifier sequences according to the present disclosure comprises (i) a variable nucleotide sequence, which is non-identical between the identifier sequences of the plurality, and (ii) an invariant nucleotide sequence, which is conserved between, and common to, the identifier sequences of the plurality. The variable nucleotide sequence may comprise or consist of 5 to 50 nucleotides, e.g. one of 10 to 30, 10 to 25, or 10 to 20 nucleotides. The invariant nucleotide sequence may comprise or consist of 2 to 25, 2 to 20, 5 to 15 or 5 to 10 nucleotides.

Invariant nucleotide sequences can be useful for the identification of identifier sequences from a given plurality of identifier sequences. For example, invariant nucleotide sequences can be useful for the identification of identifier sequences that arise from a common population of polynucleotides/vectors/cells. The use of identifier sequences comprising invariant sequences is particularly useful in embodiments of the present disclosure wherein a plurality of (e.g. 2, 3, 4, 5, 6, 7, 8 or more) polynucleotides of interest having different nucleotide sequences of interest (e.g. nucleotide sequences encoding non-identical polypeptides/nucleic acids of interest), are to be integrated into the genomic DNA of a cell. In accordance with such embodiments, invariant sequences may be used to identify an identifier sequence as being from a given polynucleotide of interest of the plurality of polynucleotides of interest.

By way of illustration, it may be desired to modify a cell to integrate 3 different polynucleotides of interest comprising non-identical nucleotide sequences of interest (e.g. polynucleotides of interest according to A, B and C) into its genomic DNA. For example, A, B and C may encode different polypeptides of interest. The method may comprise contacting a population of cells with (i) a plurality of vectors comprising a polynucleotide of interest according to A, (ii) a plurality of vectors comprising a polynucleotide of interest according to B, and (iii) a plurality of vectors comprising a polynucleotide of interest according to C.

Each polynucleotide of interest according to A of the plurality of vectors according to (i) encodes the same polypeptide of interest, but comprises a unique identifier sequence. The unique identifier sequence in turn comprises a variable nucleotide sequence (which is unique to a given polynucleotide of interest according to A in a given vector of the plurality of (i)), and an invariant nucleotide sequence, which is common to polynucleotides of interest according to A in the vectors of the plurality of (i)). Similarly, each polynucleotide of interest according to B of the plurality of vectors according to (ii) encodes the same polypeptide of interest, but comprises a unique identifier sequence which comprises a variable nucleotide sequence (unique to a given polynucleotide of interest according to B in a given vector of the plurality of (ii)), and an invariant nucleotide sequence (common to polynucleotides of interest according to B in the vectors of the plurality of (ii)). Similarly, each polynucleotide of interest according to C of the plurality of vectors according to (iii) encodes the same polypeptide of interest, but comprises a unique identifier sequence which comprises a variable nucleotide sequence (unique to a given polynucleotide of interest according to C in a given vector of the plurality of (iii)), and an invariant nucleotide sequence (common to polynucleotides of interest according to C in the vectors of the plurality of (iii)). The invariant sequences of identifier sequences of polynucleotides of interest according to A, B and C are preferably non-identical, such that the invariant nucleotide sequence of a given identifier sequence is able to identify the given identifier sequence as being an identifier sequence of a polynucleotide of interest according to A, B or C (i.e. is able to identify that the polynucleotide of interest is derived from plurality of vectors according to (i), (ii) or (iii).

It will be appreciated that the identifier sequences of the present disclosure provide for the detection and identification of polynucleotides of interest/vectors comprising the identifier sequences, by analysis of the nucleotide sequence of the identifier sequences. In preferred embodiments, a given polynucleotide of interest according to the present disclosure comprises a single identifier sequence.

Polynucleotides of interest according to the present disclosure preferably also comprise a nucleotide sequence of interest.

A nucleotide sequence of interest may be any nucleotide sequence. A nucleotide sequence may e.g. encode one or more polypeptides of interest, and/or may encode one or more nucleic acids of interest. For example, a nucleotide sequence of interest may encode an RNA of interest, e.g. an miRNA, shRNA, siRNA, etc. In some embodiments, a nucleotide sequence of interest according to the present disclosure encodes one or more polypeptides of interest. Accordingly, in some embodiments, a polynucleotide of interest according to the present disclosure comprises a nucleotide sequence encoding one or more polypeptides of interest.

A polypeptide of interest may be any polypeptide. In some embodiments, a polypeptide of interest according to the present disclosure is selected from: an antigen-binding polypeptide, an aptamer, a constituent polypeptide of an antigen-binding polypeptide complex, an antibody/antigen-binding fragment or derivative thereof, a constituent polypeptide of an antibody/antigen-binding fragment or derivative thereof, an Fc fusion protein, an anticoagulant, a blood factor, a bone morphogenetic protein, a decoy receptor for a ligand, a decoy ligand for a receptor, an enzyme, an enzyme co-factor, a growth factor, a hormone, an interferon, an interleukin, a thrombolytic, a transcription factor, an epigenetic modifier, a constituent protein of a site-specific nuclease nucleic acid editing system (e.g. a CRISPR/Cas9 system, a CRISPR/Cpf1 system, a CRISPR/C2c1 system, a CRISPR/C2c2 system, a CRISPR/C2c3 system, a ZFN system or a TALEN system), a constituent protein of a ribonucleoprotein, a viral protein (e.g. a capsid protein or a viral enzyme), or a protein useful for the production of a biomolecule.

In some embodiments, the polynucleotide of interest comprises a nucleotide sequence encoding a polypeptide, or a plurality of polypeptides, for producing polypeptide complex of interest.

In some embodiments, a polypeptide of interest is a polypeptide suitable for use in therapy or prophylaxis of a disease/condition, or a constituent polypeptide of a polypeptide complex suitable for use in therapy or prophylaxis of a disease/condition. A polypeptide/polypeptide complex suitable for use in therapy or prophylaxis of a disease/condition may be any polypeptide/polypeptide complex whose administration is useful for the treatment or prevention of a disease/condition.

In some embodiments, a polypeptide of interest is an antigen-binding polypeptide, or a constituent polypeptide of an antigen-binding polypeptide complex. Antigen-binding polypeptides/polypeptide complexes include antibodies (i.e. immunoglobulins (Igs)) and antigen-binding fragments of antibodies. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH, etc.). Antigen-binding fragments of antibodies include e.g. Fv, Fab, F (ab′) 2 and F(ab′) fragments.

Antigen-binding polypeptides/polypeptide complexes also include e.g. a peptide aptamers, thioredoxins, monobodies, anticalins, Kunitz domains, avimers, knottins, fynomers, atrimers, DARPins, affibodies, nanobodies (i.e. a single-domain antibodies (sdAbs)), affilins, armadillo repeat proteins (ArmRPs), OBodies and fibronectins-reviewed e.g. in Reverdatto et al., Curr Top Med Chem. 2015; 15 (12): 1082-1101, which is hereby incorporated by reference in its entirety (see also e.g. Boersma et al., J Biol Chem (2011) 286:41273-85 and Emanuel et al., Mabs (2011) 3:38-48).

In some embodiments, a polypeptide of interest is a multispecific antigen-binding polypeptide, or a constituent polypeptide of a multispecific antigen-binding polypeptide complex. By ‘multispecific’ it is meant that the antigen-binding polypeptide/polypeptide complex displays specific binding to more than one target antigen (e.g. one of 2, 3, 4, 5, 6 or more target antigens). In some embodiments, a multispecific antigen-binding polypeptide/polypeptide complex is a bispecific antigen-binding polypeptide/polypeptide complex.

In some embodiments, a multispecific antigen-binding polypeptide/polypeptide complex comprises at least two, non-identical antigen-binding domains. The antigen-binding domains of a multispecific antigen-binding polypeptide/polypeptide complex may comprise a variable heavy chain region (VH) and a variable light chain region (VL), e.g. of an antibody capable of binding to a given target antigen.

In some embodiments, a polypeptide of interest is a detectable polypeptide or a polypeptide having detectable activity. A detectable polypeptide may be or comprise a fluorescent polypeptide. Fluorescent polypeptides include green fluorescent protein and variants thereof (e.g. enhanced green fluorescent protein), yellow fluorescent protein (e.g. citrine), red fluorescent protein and variants thereof (e.g. mOrange, mCherry), blue fluorescent protein and variants thereof (e.g. TagBFP), cyan fluorescent protein and variants thereof (e.g. mTurquoise, cerulean), allophycocyanin, phycocyanin, phycoerythrin and phycoerythrocyanin. A detectable polypeptide may be or comprise an epitope tag. Epitope tags include e.g. His, (e.g. 6×His; SEQ ID NO:23), FLAG, c-Myc, StrepTag, haemagglutinin, E-tag, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol). A polypeptide having detectable activity may be or comprise an enzymatic moiety. Enzymatic moieties include e.g. luciferases, glucose oxidases, galactosidases (e.g. beta-galactosidase), glucorinidases, phosphatases (e.g. alkaline phosphatase), peroxidases (e.g. horseradish peroxidase) and cholinesterases.

In some embodiments, polynucleotides of interest according to the present disclosure comprise a nucleotide sequence encoding more than one polypeptide of interest. In some embodiments, a polynucleotide of interest encodes one of 2, 3, 4, 5, 6, 7, 8, 9, 10 or more polypeptides of interest. In embodiments wherein a polynucleotide of interest encodes more than one polypeptide of interest, it will be appreciated that each polypeptide of interest is independently a polypeptide of interest according to the present disclosure.

In some embodiments where a polynucleotide of interest encodes more than one polypeptide of interest, the plural polypeptides of interest are capable of associating to form a polypeptide complex. That is, in some embodiments, a polynucleotide of interest encodes more than one polypeptide of interest, and the polypeptides of interest are constituent polypeptides of a polypeptide complex. In accordance with such embodiments, following expression of the polypeptides of interest from the polynucleotide of interest in a cell comprising the polynucleotide of interest, the polypeptides of interest may associate with one another to form the polypeptide complex.

In some embodiments, a polynucleotide of interest encodes the constituent polypeptides of an antigen-binding polypeptide complex. In some embodiments, a polynucleotide of interest encodes the constituent polypeptides of a multispecific (e.g. bispecific) antigen-binding polypeptide complex.

In some embodiments, a polynucleotide of interest may additionally comprise one or more non-polypeptide-encoding nucleotide sequence(s). Non-polypeptide-encoding nucleotide sequence(s) include e.g. promoter, enhancer, termination codon, 5′ cap, 5′ UTR, 3′ UTR and/or polyadenylation signal sequences.

In some embodiments, the polynucleotide of interest comprises a 5′ UTR 5′ to (i.e. upstream of, in the context of the nucleotide sequence of the polynucleotide) a start codon.

In some embodiments, the polynucleotide of interest comprises a 3′ UTR 3′ to (i.e. downstream of, in the context of the nucleotide sequence of the polynucleotide) a stop codon. In some embodiments, the polynucleotide comprises a 3′ UTR 5′ to a polyadenylation signal sequence. In some embodiments, the polynucleotide comprises a 3′ UTR 3′ to a stop codon and 5′ to a polyadenylation signal sequence.

In some embodiments, the polynucleotide of interest further comprises nucleotide sequence(s) for expression of the one or more polypeptides encoded by the polynucleotide of interest. In some embodiments, the polynucleotide of interest comprises one or more regulatory nucleotide sequence(s) operably-linked to a nucleotide sequence encoding one or more polypeptides of interest according to the present disclosure. Regulatory nucleotide sequence(s) include promoter sequences and enhancer sequences for driving and/or increasing expression of the one or more polypeptides from the polynucleotide of interest. The term ‘operably-linked’ may include the situation where the nucleotide sequence nucleic acid encoding one or more polypeptides of interest and regulatory nucleotide sequence(s) (e.g. a promoter sequence and/or enhancer sequence) are covalently linked in such a way as to place the expression of nucleotide sequence nucleic acid encoding one or more polypeptides of interest under the influence or control of the regulatory nucleotide sequence(s) (thereby forming an expression cassette). Thus, a regulatory nucleotide sequence is operably-linked to the nucleotide sequence encoding one or more polypeptides of interest if the regulatory nucleotide sequence is capable of effecting transcription of the nucleotide sequence. The resulting transcript(s) may then be translated into the polypeptide(s) of interest.

Any suitable regulatory nucleotide sequence(s) may be employed in a polynucleotide of interest according to the present disclosure. In some embodiments, a promoter sequence is a promoter sequence that drives expression in a mammalian cell. A promoter may provide for constitutive expression of the nucleic acid under the control of the promoter, or may provide for inducible expression of the nucleic acid under the control of the promoter (e.g. in response to a given chemical). Suitable promoter sequences for expression in mammalian cells are well known to the skilled person, and examples of such promoters are described e.g. in Chen et al., PLOS ONE (2011) 6 (8): e23376, which is hereby incorporated by reference in its entirety. Such promoter sequences include the chicken β-actin promoter, human EF1a promoter, mouse PGK promoter, human ubiquitin C promoter, MC1 promoter, the immediate early enhancer of human CMV, deletion derivatives of the CMV promoter, e.g. CMVd1, the CMV immediate early enhancer/chicken β-actin promoter/rabbit β-globin intron composite promoter (CAG). In some embodiments, a promoter sequence is a CMV promoter or a SV40 promoter.

In some embodiments, the polynucleotide of interest comprises a nucleotide sequence encoding a selectable marker, to facilitate identification and/or selection of cells comprising/expressing the polynucleotide of interest. Selectable markers include proteins that confer resistance to cytotoxic compounds, e.g. antibiotics or other toxins, e.g., puromycin, blasticidin, ampicillin, neomycin, methotrexate, or tetracycline, and proteins that complement auxotrophic deficiencies. By way of illustration, in the experimental examples of the present disclosure, the polynucleotide of interest comprises a nucleotide sequence encoding puromycin N-acetyltransferase, which confers resistance to puromycin.

Aspects and embodiments of the present disclosure relate to a plurality of polynucleotides of interest. In accordance with such aspects and embodiments, the identifier sequences of individual polynucleotides of interest of the plurality of polynucleotides are non-identical. In preferred embodiments, an identifier sequence of a polynucleotide of interest within a plurality of polynucleotides of interest is non-identical to an identifier sequence of any other polynucleotide of interest within the plurality of polynucleotides of interest. That is, each polynucleotide of interest within a plurality of polynucleotides of interest preferably comprises a unique identifier sequence (i.e. with respect to the identifier sequences of the other polynucleotides of interest of the plurality).

In preferred embodiments, polynucleotides of interest within a given plurality of polynucleotides of interest comprise a nucleotide sequence encoding the same polypeptide(s) as the other polynucleotides of interest within the plurality thereof.

In preferred embodiments, within a given plurality of polynucleotides of interest (i) each polynucleotide of interest comprises a unique identifier sequence, and (ii) the polynucleotides of interest of the plurality of polynucleotides of interest encode the same polypeptide(s) of interest.

### Polynucleotides Comprising Polynucleotides of Interest, Vectors

In some embodiments, a polynucleotide of interest according to the present disclosure is provided within (i.e. comprised in) a larger polynucleotide. That is, the nucleotide sequence of the polynucleotide of interest is contained within, and is a subsequence of, the larger polynucleotide. In some embodiments, the polynucleotide comprising a polynucleotide of interest is a vector. In some embodiments, the polynucleotide comprising a polynucleotide of interest is in turn comprised in a vector.

Herein, a ‘vector’ refers to a nucleic acid molecule used as a vehicle to transfer exogenous nucleic acid into a cell. A vector according to the present disclosure comprises a polynucleotide of interest according to the present disclosure, and may facilitate delivery of the polynucleotide of interest into a cell. Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol. (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, a polynucleotide (e.g. a vector) comprising a polynucleotide of interest comprises nucleotide sequence(s) facilitating integration of the polynucleotide of interest into the genomic DNA of a cell into which it is introduced. For example, a polynucleotide/vector comprising a polynucleotide of interest may comprise nucleotide sequences facilitating integration of the polynucleotide of interest into the genomic DNA of a cell into which it is introduced, by homologous recombination. In some embodiments, nucleotide sequence(s) facilitating integration of the polynucleotide of interest into the genomic DNA of a cell into which it is introduced are also provided on a polynucleotide other than the polynucleotide comprising the polynucleotide of interest.

In preferred embodiments, nucleotide sequence(s) facilitating integration of the polynucleotide of interest into the genomic DNA of a cell into which the polynucleotide of interest is introduced provide for targeted integration of the polynucleotide of interest. ‘Targeted integration’ or ‘site-specific integration’ of a given nucleotide sequence into an acceptor nucleotide sequence generally refers to integration at a particular location of the acceptor nucleotide sequence, and is distinguished from random/non-specific integration. The site of integration into the acceptor nucleotide sequence may be controlled by nucleotide sequences facilitating integration comprised in one or both of the nucleotide sequence to be integrated, and/or the acceptor nucleotide sequence.

In preferred embodiments, nucleotide sequence(s) facilitating integration of the polynucleotide of interest into the genomic DNA of a cell into which the polynucleotide of interest is introduced provide for integration of a single copy of a given polynucleotide of interest according to the present disclosure. That is, in preferred embodiments, only a single copy of a given polynucleotide of interest according to the present disclosure is integrated into the genomic DNA of a given cell into which the polynucleotide of interest is introduced. Thus, in accordance with such embodiments, a cell having the polynucleotide of interest integrated into its genomic DNA comprises a single identifier sequence for the given polynucleotide of interest, unique to that cell.

To be clear, aspects and embodiments of the present disclosure contemplate integration of multiple, different polynucleotides of interest, comprising non-identical nucleotide sequences of interest (e.g. encoding different polypeptides/nucleic acids). In accordance with such aspects and embodiments, a single copy of a given polynucleotide of interest (i.e. of the multitude) is still integrated, but additional, different polynucleotides of interest (e.g. comprising a different nucleotide sequence of interest, relative to the nucleotide sequence of interest of the given polynucleotide of interest) are also integrated.

A given polynucleotide (e.g. a vector) comprising a polynucleotide of interest according to the present disclosure preferably comprises a single copy of a given polynucleotide of interest, and thus a single identifier sequence for the given polynucleotide of interest.

In some embodiments, a polynucleotide (e.g. a vector) comprising a polynucleotide of interest comprises nucleotide sequence(s) providing for integration of the polynucleotide of interest into the genomic DNA of the cell into which the polynucleotide of interest is to be introduced. In some embodiments, nucleotide sequence(s) providing for integration of the polynucleotide of interest are additionally, or alternatively, provided on a polynucleotide (e.g. a vector) that does not comprise the polynucleotide of interest.

In some embodiments, nucleotide sequence(s) providing for integration of the polynucleotide of interest into the genomic DNA of the cell into which the polynucleotide of interest is to be introduced provide for integration of the polynucleotide of interest into the genomic DNA of the cell via recombinase-mediated cassette exchange (RMCE).

Site-specific recombinase (SSR) systems for introduction of a given nucleotide sequence into an acceptor nucleotide sequence via RMCE are well known in the art. Such SSR systems include e.g. Cre-Lox, Flp-FRT, and Dre-rox systems, and are described e.g. in Tian and Zhou, J Biol Chem. (2021) 296:100509 and Turan et al., Gene (2013) 515 (1): 1-27, both of which are hereby incorporated by reference in their entirety. Variants of such SSR systems and other SSR systems are also well known in the art and can similarly be employed. The use of RMCE to introduce a polynucleotide of interest encoding a polypeptide(s) of interest into the genomic DNA of a cell is described e.g. in Srirangan et al., Crit Rev Biotechnol. (2020) 40 (6): 833-851, which is hereby incorporated by reference in its entirety.

In some embodiments, a polynucleotide (e.g. a vector) comprising a polynucleotide of interest comprises one or more target sequences for a recombinase (e.g. Cre recombinase, Flp recombinase, Dre recombinase, etc.). In some embodiments, the polynucleotide/vector comprising a polynucleotide of interest comprises target sequences for a recombinase flanking the polynucleotide of interest.

In some embodiments, a target sequence for a recombinase according to the present disclosure is a lox sequence, e.g. a loxP, loxFAS or L3 sequence (homologous recombination between compatible lox sequences is catalyzed by Cre recombinase). In some embodiments, a target sequence for a recombinase is a FRT sequence (homologous recombination between such sequences is catalyzed by Flp recombinase). In some embodiments, a target sequence for a recombinase is a rox sequence (homologous recombination between such sequences is catalyzed by Dre recombinase).

It will be appreciated that nucleotide sequence(s) facilitating integration of a polynucleotide of interest into the genomic DNA of a cell into which a polynucleotide of interest is to be introduced are selected to be suitable for (i.e. compatible with) integration of the polynucleotide of interest into the genomic DNA of that cell. That is, the nucleotide sequence(s) facilitating integration of a polynucleotide of interest are complementary to nucleotide sequence(s) providing for targeted integration of the polynucleotide of interest in the genomic DNA of the cell into which the polynucleotide/vector is to be introduced. For example, they may be selected for compatibility with nucleotide sequence(s) facilitating integration of a polynucleotide of interest provided in the genomic DNA of the cell into which the polynucleotide of interest is to be integrated. By way of illustration, where the genomic DNA of a cell into which a polynucleotide of interest is to be integrated comprises one or more lox sequences, the polynucleotide/vector comprising a polynucleotide of interest may comprise lox sequences flanking the nucleotide sequence of a polynucleotide of interest.

In some embodiments, particularly embodiments in which the polynucleotide of interest is to be integrated into the genomic DNA of a cell via RMCE, the polynucleotide/vector comprising a polynucleotide of interest may further comprise a nucleotide sequence encoding the relevant recombinase. For example, in some embodiments wherein the polynucleotide/vector comprising a polynucleotide of interest comprises target sequences for a given recombinase flanking the nucleotide sequence of a polynucleotide of interest, the polynucleotide/vector may further comprise a nucleotide sequence encoding the given recombinase. By way of illustration, where a polynucleotide/vector comprising a polynucleotide of interest comprises lox sequences flanking the nucleotide sequence of a polynucleotide of interest, the polynucleotide/vector may further comprise a nucleotide sequence encoding Cre recombinase.

Aspects and embodiments of the present disclosure relate to a plurality of polynucleotides (e.g. a plurality of vectors) comprising a polynucleotide of interest. In accordance with such aspects and embodiments, the identifier sequences of individual polynucleotides/vectors comprising a polynucleotide of interest of the plurality are non-identical. In some embodiments, the polynucleotides of interest differ only in the nucleotide sequence of their identifier sequences.

In some embodiments, the identifier sequences of individual polynucleotides/vectors of the plurality comprise variable nucleotide sequences which are non-identical with respect to one another. In some embodiments, the identifier sequences of individual polynucleotides/vectors of the plurality comprise (i) variable nucleotide sequences, which are non-identical with respect to one another, and (ii) invariant nucleotide sequences, which are conserved between, and common to, the identifier sequences of individual polynucleotides/vectors of the plurality.

In preferred embodiments, an identifier sequence of a polynucleotide of interest comprised in a polynucleotide/vector comprising a polynucleotide of interest within a plurality thereof is non-identical to an identifier sequence of any other polynucleotide of interest comprised in a polynucleotide/vector comprising a polynucleotide of interest within the plurality thereof. That is, each polynucleotide/vector comprising a polynucleotide of interest within a plurality thereof preferably comprises a unique identifier sequence (i.e. with respect to the identifier sequences of the other polynucleotides/vectors of the plurality).

In some embodiments, for a given plurality of polynucleotides (e.g. a plurality of vectors) comprising a polynucleotide of interest, each individual polynucleotide/vector of the plurality comprises a polynucleotide of interest comprising the same nucleotide sequence of interest. In some embodiments, for a given plurality of polynucleotides (e.g. a plurality of vectors) comprising a polynucleotide of interest, each individual polynucleotide/vector of the plurality comprises a polynucleotide of interest encoding the same polypeptide(s).

In preferred embodiments, within a given plurality of polynucleotides (e.g. a plurality of vectors) comprising a polynucleotide of interest (i) each polynucleotide/vector of the plurality comprises a polynucleotide of interest having a unique identifier sequence, and (ii) the polynucleotides of interest comprised within the polynucleotides/vectors of the plurality comprise the same nucleotide sequence of interest. In preferred embodiments, within a given plurality of polynucleotides (e.g. a plurality of vectors) comprising a polynucleotide of interest (i) each polynucleotide/vector of the plurality comprises a polynucleotide of interest having a unique identifier sequence, and (ii) the polynucleotides of interest comprised within the polynucleotides/vectors of the plurality encode the same polypeptide(s) of interest.

In some embodiments, within a given plurality of polynucleotides (e.g. a plurality of vectors) comprising a polynucleotide of interest (i) each polynucleotide/vector of the plurality comprises a polynucleotide of interest having a unique identifier sequence, which comprises (a) a unique variable nucleotide sequence, and (b) an invariant sequence which is conserved between, and common to, the identifier sequences of individual polynucleotides/vectors of the plurality; and (ii) the polynucleotides of interest comprised within the polynucleotides/vectors of the plurality comprise the same nucleotide sequence of interest. In some embodiments, within a given plurality of polynucleotides (e.g. a plurality of vectors) comprising a polynucleotide of interest (i) each polynucleotide/vector of the plurality comprises a polynucleotide of interest having a unique identifier sequence, which comprises (a) a unique variable nucleotide sequence, and (b) an invariant sequence which is conserved between, and common to, the identifier sequences of individual polynucleotides/vectors of the plurality; and (ii) the polynucleotides of interest comprised within the polynucleotides/vectors of the plurality encode the same polypeptide(s) of interest.

Aspects and embodiments of the present disclosure contemplate the introduction of a plurality (e.g. 2, 3, 4, 5, 6, 7, 8 or more) of polynucleotides of interest into the genomic DNA of a cell, wherein the individual polynucleotides of interest of the plurality of polynucleotides of interest comprise different nucleotide sequences of interest (e.g. nucleotide sequences encoding non-identical polypeptides/nucleic acids of interest). For example, integration of at least two different polynucleotides of interest is contemplated: a first polynucleotide of interest comprising a first nucleotide sequence of interest, and a second polynucleotide of interest comprising a second nucleotide sequence of interest, wherein the first nucleotide sequence of interest and the second nucleotide sequence of interest are non-identical.

Accordingly, in some aspects and embodiments, the present disclosure provides multiple (e.g. 2, 3, 4, 5, 6, 7, 8 or more) pluralities of polynucleotides (e.g. multiple pluralities of vectors), each comprising a polynucleotide of interest according to the present disclosure. In accordance with such aspects and embodiments, the pluralities of polynucleotides within the larger group thereof may comprise non-identical nucleotide sequences of interest. In some embodiments, the pluralities of polynucleotides within the larger group thereof encode non-identical polypeptide(s) or nucleic acid(s) of interest.

By way of illustration, 3 different pluralities of polynucleotides (e.g. 3 different pluralities of vectors) may be provided: a plurality of polynucleotides comprising a polynucleotide of interest according to A, a plurality of polynucleotides comprising a polynucleotide of interest according to B, and a plurality of polynucleotides comprising a polynucleotide of interest according to C. Polynucleotides of interest according to A provided within the plurality of polynucleotides comprising a polynucleotide of interest according to A encode the same polypeptide(s) of interest, but comprise non-identical identifier sequences. Similarly, polynucleotides of interest according to B provided within the plurality of polynucleotides comprising a polynucleotide of interest according to B encode the same polypeptide(s) of interest, but comprise non-identical identifier sequences. Similarly, polynucleotides of interest according to C provided within the plurality of polynucleotides comprising a polynucleotide of interest according to C encode the same polypeptide(s) of interest, but comprise non-identical identifier sequences.

Polynucleotides of interest according to A, B and C may encode different polypeptide(s) of interest. That is, a polynucleotide of interest according to A may encode a polypeptide of interest which is non-identical to the polypeptide(s) of interest encoded by a polynucleotide of interest according to B, and which is non-identical to the polypeptide(s) of interest encoded by a polynucleotide of interest according to C. Similarly, a polynucleotide of interest according to B may encode a polypeptide of interest which is non-identical to the polypeptide(s) of interest encoded by a polynucleotide of interest according to A, and which is non-identical to the polypeptide(s) of interest encoded by a polynucleotide of interest according to C. Similarly, a polynucleotide of interest according to C may encode a polypeptide of interest which is non-identical to the polypeptide(s) of interest encoded by a polynucleotide of interest according to A, and which is non-identical to the polypeptide(s) of interest encoded by a polynucleotide of interest according to B.

In accordance with such aspects and embodiments, the identifier sequences of a given plurality of polynucleotides within the larger group thereof may comprise the same invariant nucleotide sequence (in addition to their unique variable nucleotide sequence). The invariant nucleotide sequence is thus able to serve to identify an identifier sequence from a given plurality of polynucleotides (from within the larger group thereof).

By way of illustration, 3 different pluralities of polynucleotides (e.g. 3 different pluralities of vectors) may be provided: a plurality of polynucleotides comprising a polynucleotide of interest according to A, a plurality of polynucleotides comprising a polynucleotide of interest according to B, and a plurality of polynucleotides comprising a polynucleotide of interest according to C. Polynucleotides of interest according to A may each comprise an identifier sequence comprising the same invariant nucleotide sequence, polynucleotides of interest according to B may each comprise an identifier sequence comprising the same invariant nucleotide sequence, and polynucleotides of interest according to C may each comprise an identifier sequence comprising the same invariant nucleotide sequence. The invariant nucleotide sequence of a polynucleotide of interest according to A is non-identical to the invariant nucleotide sequence of a polynucleotide of interest according to B, and is also non-identical to the invariant nucleotide sequence of a polynucleotide of interest according to C. Similarly, the invariant nucleotide sequence of a polynucleotide of interest according to B is non-identical to the invariant nucleotide sequence of a polynucleotide of interest according to A, and is also non-identical to the invariant nucleotide sequence of a polynucleotide of interest according to C. Similarly, the invariant nucleotide sequence of a polynucleotide of interest according to C is non-identical to the invariant nucleotide sequence of a polynucleotide of interest according to A, and is also non-identical to the invariant nucleotide sequence of a polynucleotide of interest according to B.

### Cells

The present disclosure relates to cells having a polynucleotide of interest according to the present disclosure integrated into their genomic DNA. The present disclosure also relates to cells into which a polynucleotide of interest according to the present disclosure is to be introduced.

A cell according to the present disclosure may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NS0 and BHK cells. In preferred embodiments, the cell is, or is derived from, a CHO cell.

A cell into which a polynucleotide of interest according to the present disclosure is to be introduced (i.e. prior to such introduction) lacks a polynucleotide of interest according to the present disclosure. In some embodiments, the genomic DNA of such cells comprises nucleotide sequence(s) facilitating integration of the polynucleotide of interest into the genomic DNA (i.e. following introduction of the polynucleotide of interest into the cell).

It will be appreciated that in embodiments wherein the genomic DNA of a cell according to the present disclosure comprises nucleotide sequence(s) facilitating integration of a polynucleotide of interest, the sequence(s) are selected to be suitable for (i.e. compatible with) integration of the polynucleotide of interest provided on a polynucleotide (e.g. a vector) comprising a polynucleotide of interest according to the present disclosure. That is, the nucleotide sequence(s) of the genomic DNA of the cell facilitating integration of a polynucleotide of interest are complementary to nucleotide sequence(s) for facilitating integration of the polynucleotide of interest provided in the polynucleotide/vector comprising a polynucleotide of interest. For example, a cell into which a polynucleotide of interest is to be integrated may comprise nucleotide sequence(s) facilitating integration of a polynucleotide of interest into its genomic DNA by homologous recombination (i.e. following introduction of the polynucleotide of interest into the cell).

In preferred embodiments, the genomic DNA of the cell into which a polynucleotide of interest is to be integrated comprises nucleotide sequence(s) providing for targeted integration of the polynucleotide of interest (i.e. following introduction of a polynucleotide/vector comprising a polynucleotide of interest into the cell). An acceptor nucleotide sequence providing for targeted integration of a nucleotide sequence to be integrated may also be referred to in the art as a ‘landing pad’. Accordingly, in some embodiments, the genomic DNA of a cell into which a polynucleotide of interest according to the present disclosure is to be integrated comprises a landing pad for the polynucleotide of interest.

It will be appreciated that in embodiments wherein the genomic DNA of a cell according to the present disclosure comprises nucleotide sequence(s) facilitating integration of a polynucleotide of interest, the sequence(s) are selected to be suitable for (i.e. compatible with) integration of the polynucleotide of interest provided on a polynucleotide (e.g. a vector) comprising a polynucleotide of interest according to the present disclosure. That is, the nucleotide sequence(s) of the genomic DNA of the cell facilitating integration of a polynucleotide of interest are complementary to nucleotide sequence(s) for facilitating integration of the polynucleotide of interest provided in the polynucleotide/vector comprising a polynucleotide of interest. For example, a cell into which a polynucleotide of interest is to be integrated may comprise nucleotide sequence(s) facilitating integration of a polynucleotide of interest into its genomic DNA by homologous recombination (i.e. following introduction of the polynucleotide of interest into the cell).

In preferred embodiments, the genomic DNA of the cell into which a polynucleotide of interest is to be integrated comprises a nucleotide sequence providing for integration of a single copy of a given polynucleotide of interest according to the present disclosure. A nucleotide sequence of an acceptor nucleotide sequence providing for integration of a single copy of a nucleotide sequence to be integrated may also be referred to as a ‘single-copy landing pad’. Accordingly, in some embodiments, the genomic DNA of a cell into which a polynucleotide of interest according to the present disclosure is to be integrated comprises a single-copy landing pad for the polynucleotide of interest.

Thus, following integration of the polynucleotide of interest into the genomic DNA of a cell in accordance with the present disclosure, the cell preferably comprises only a single copy of a polynucleotide of interest, and similarly a single identifier sequence, which is unique to that cell.

In some embodiments, the genomic DNA of a cell into which a polynucleotide of interest is to be integrated comprises nucleotide sequence(s) providing for integration of the polynucleotide of interest into the genomic DNA via RMCE.

In some embodiments, the genomic DNA of a cell into which a polynucleotide of interest is to be integrated comprises one or more target sequences for a recombinase (e.g. Cre recombinase, Flp recombinase, Dre recombinase, etc.). A target sequence for a recombinase may be e.g. a lox sequence (e.g. a loxP (also known as ‘L2’), loxFAS or L3 sequence), a FRT sequence or a rox sequence.

It will be appreciated that in embodiments wherein a cell into which a polynucleotide of interest is to be integrated comprises nucleotide sequence(s) facilitating integration of the polynucleotide of interest into the genomic DNA (i.e. following introduction of the polynucleotide of interest into the cell), the nucleotide sequence(s) facilitating integration are selected to be suitable for (i.e. compatible with) integration of the polynucleotide of interest into the genomic DNA of that cell. For example, they may be selected for compatibility with nucleotide sequence(s) facilitating integration of a polynucleotide of interest provided in a polynucleotide/vector comprising a polynucleotide of interest. By way of illustration, where a polynucleotide/vector comprising a polynucleotide of interest to be integrated comprises lox sequences flanking the nucleotide sequence of a polynucleotide of interest, the genomic DNA of the cell into which a polynucleotide of interest is to be integrated may comprise one or more lox sequences.

In some embodiments, the genomic DNA of a cell into which a polynucleotide of interest according to the present disclosure is to be integrated comprises nucleotide sequence(s) facilitating integration of a plurality of (e.g. 2, 3, 4, 5, 6, 7, 8 or more) polynucleotides of interest (wherein polynucleotides of interest of the plurality have different nucleotide sequences of interest, e.g. nucleotide sequences encoding non-identical polypeptides/nucleic acids of interest). Following integration of the different polynucleotides of interest into the genomic DNA of such a cell, the cell comprises only a single copy of each of the different polynucleotides of interest, and accordingly a unique profile of identifier sequences (i.e. derived from the identifier sequences of the different polynucleotides of interest of the plurality).

It will be appreciated that in embodiments wherein the genomic DNA of a cell according to the present disclosure comprises nucleotide sequence(s) facilitating integration of plural, different polynucleotides of interest, the sequence(s) are selected to be suitable for (i.e. compatible with) integration of the different polynucleotides of interest. That is, the nucleotide sequence(s) of the genomic DNA of the cell facilitating integration of the plurality of different polynucleotides of interest are complementary to nucleotide sequence(s) for facilitating integration of the different polynucleotides of interest provided in the polynucleotides/vectors comprising the different polynucleotides of interest.

For example, a cell into which the plurality of different polynucleotides of interest (e.g. nucleotide sequences encoding non-identical polypeptides/nucleic acids of interest) is to be integrated may comprise nucleotide sequence(s) facilitating integration of the various different polynucleotides of interest into its genomic DNA by homologous recombination (i.e. following introduction of the polynucleotides of interest into the cell).

In some embodiments, the genomic DNA of a cell into which a polynucleotide of interest according to the present disclosure is to be integrated comprises more than one single-copy landing pad. In some embodiments, the genomic DNA of a cell into which a polynucleotide of interest according to the present disclosure is to be integrated comprises 2, 3, 4, 5, 6, 7, 8 or more single-copy landing pads.

Such cells comprising a plurality of single-copy landing pads may be used to produce cells comprising a plurality of (e.g. 2, 3, 4, 5, 6, 7, 8 or more) integrated polynucleotides of interest, wherein each polynucleotide of interest of the plurality has a different nucleotide sequence of interest (e.g. encode non-identical polypeptides/nucleic acids of interest). Following integration of the different polynucleotides of interest into the genomic DNA of such a cell, the cell preferably comprises only a single copy of each of the different polynucleotides of interest, and accordingly a unique profile of identifier sequences (i.e. derived from the identifier sequences of the different polynucleotides of interest of the plurality).

By way of illustration, a cell may comprise e.g. 3 different single-copy landing pads, which may be used for the targeted integration of 3 different polynucleotides of interest (e.g. polynucleotides of interest according to A, B and C), wherein polynucleotides of interest according to A, B and C encode different polypeptides of interest. Polynucleotides of interest according to A, B and C may be integrated into the genomic DNA of the cell by contacting a population of such cells with (i) a plurality of vectors comprising a nucleotide sequence providing for targeted integration of a polynucleotide of interest (comprising an identifier sequence) according to A, (ii) a plurality of vectors comprising a nucleotide sequence providing for targeted integration of a polynucleotide of interest (comprising an identifier sequence) according to B, and (iii) a plurality of vectors comprising a nucleotide sequence providing for targeted integration of a polynucleotide of interest (comprising an identifier sequence) according to C; under conditions suitable for introducing the vectors into the cells. The vectors of each plurality of vectors comprise a polynucleotide of interest having a unique identifier sequence; that is—with reference to the preceding sentence—no two vectors of (i) have the same identifier sequence, no two vectors of (ii) have the same identifier sequence, and no two vectors of (iii) have the same identifier sequence. Following integration, the cell comprises genomic DNA comprising a single copy of a polynucleotide of interest according to A, a single copy of a polynucleotide of interest according to B and a single copy of a polynucleotide of interest according to C. The cell also comprises a unique profile of identifier sequences, formed of the identifier sequence from the integrated polynucleotide of interest according to A, the identifier sequence from the integrated polynucleotide of interest according to B, and the identifier sequence from the integrated polynucleotide of interest according to C.

In some embodiments, the genomic DNA of a cell into which a plurality of different polynucleotides of interest is to be integrated comprises nucleotide sequence(s) providing for integration of the different polynucleotides of interest into the genomic DNA via RMCE.

In some embodiments, the genomic DNA of a cell into which a plurality of different polynucleotides of interest is to be integrated comprises target sequences for a plurality of different recombinases. By way of illustration, a cell may comprise target sequences for Cre recombinase, target sequences for Flp recombinase, and target sequences for Dre recombinase.

In some embodiments, the genomic DNA of a cell into which one or more polynucleotides of interest according to the present disclosure are to be integrated comprises three different target sequences for a recombinase (i.e. target sequences 1, 2 and 3). Target sequences 1, 2 and 3 may be provided in an arrangement wherein target sequence 3 is provided between target sequence 1 and target sequence 2. Target sequences 1, 2 and 3 preferably do not cross-react with one another (that is, they are not compatible for recombination between one another). In some embodiments, a nucleotide sequence encoding a selectable marker is provided between target sequence 1 and target sequence 3. In some embodiments, each of target sequences 1, 2 and 3 are target sequences for the same recombinase. In some embodiments, each of target sequences 1, 2 and 3 are target sequences for Cre recombinase. In some embodiments, one of target sequences 1, 2 and 3 is an L3 sequence, another of target sequences 1, 2 and 3 is a loxP sequence, and the remaining target sequence of target sequences 1, 2 and 3 is a loxFAS sequence.

In some embodiments, target sequence 1 is an L3 sequence, target sequence 2 is a loxP sequence, and target sequence 3 is a loxFAS sequence.

Such cells may be employed in a ‘two-vector’ strategy for targeted integration of one or more polynucleotides of interest into their genomic DNA, e.g. with: (i) a first polynucleotide (e.g. a vector) comprising (from 5′ to 3′): (a) a first target sequence for a recombinase which is compatible for recombination with target sequence 1 of the genomic DNA of the cell (e.g. the same target sequence for a recombinase as target 1), (b) a promoter and start codon, and (c) a second target sequence for a recombinase which is compatible for recombination with target sequence 3 of the genomic DNA of the cell (e.g. the same target sequence for a recombinase as target 3); and (ii) a second polynucleotide (e.g. a vector) comprising: (a) a third target sequence for a recombinase which is compatible for recombination with target sequence 3 of the genomic DNA of the cell (e.g. the same target sequence for a recombinase as target 3), (b) a nucleotide sequence encoding a selectable marker (which is different to the selectable marker encoded by the nucleotide sequence provided between target sequence 1 and target sequence 3 of the genomic DNA of the cell, in embodiments where the genomic DNA of the cell comprises such a selectable nucleotide sequence) lacking a start codon, and (c) a fourth target sequence for a recombinase which is compatible for recombination with target sequence 2 of the genomic DNA of the cell (e.g. the same target sequence for a recombinase as target 2).

Targeted integration may be achieved by dual RMCE, in which the promoter and start codon of the first polynucleotide are integrated via RCME between the first target sequence for a recombinase and target sequence 1 of the genomic DNA of the cell, and between the second target sequence for a recombinase and target sequence 3 of the genomic DNA of the cell; and in which the nucleotide sequence encoding the selectable marker lacking a start codon is integrated via RCME between the third target sequence for a recombinase and target sequence 3 of the genomic DNA of the cell, and between the fourth target sequence for a recombinase and target sequence 2 of the genomic DNA of the cell. Following dual RMCE, the genomic DNA of the cell has integrated nucleotide sequence (b) of the first polynucleotide, and nucleotide sequence (b) of the second polynucleotide. Cells in which dual RMCE has taken place may be identified via appropriate selection for expression of the selectable marker, which will only be expressed by cells having integrated both nucleotide sequence (b) of the first polynucleotide and nucleotide sequence (b) of the second polynucleotide.

In accordance with such embodiments, the first polynucleotide may comprise a nucleotide sequence of interest according to the present disclosure (e.g. encoding one or more polypeptides of interest) in between the first target sequence for a recombinase and the second target sequence for a recombinase (e.g. 5′ to the promoter and start codon); and/or the second polynucleotide may comprise a nucleotide sequence of interest according to the present disclosure (e.g. encoding one or more polypeptides of interest) in between the third target sequence for a recombinase and the fourth target sequence for a recombinase (e.g. 3′ to the nucleotide sequence encoding the selectable marker lacking a start codon). In accordance with such embodiments, the first polynucleotide may comprise an identifier sequence according to the present disclosure in between the first target sequence for a recombinase and the second target sequence for a recombinase (e.g. 5′ to the promoter and start codon), or the second polynucleotide may comprise an identifier sequence according to the present disclosure in between the third target sequence for a recombinase and the fourth target sequence for a recombinase (e.g. 3′ to the nucleotide sequence encoding the selectable marker lacking a start codon).

It will be appreciated that the present disclosure also provides a first polynucleotide (e.g. vector) according to an embodiment described in any one of the preceding three paragraphs, and also provides a second polynucleotide (e.g. vector) according to an embodiment described in any one of the preceding three paragraphs.

In some embodiments, a cell into which a polynucleotide of interest according to the present disclosure is to be introduced is a targeted integration (TI) host cell according to any embodiment described in WO 2019/126634 A2, which is hereby incorporated by reference in its entirety. In some embodiments, a cell according to the present disclosure is a targeted integration (TI) host cell comprising an exogenous nucleotide sequence integrated at an integration site within a specific locus of the genome of the host cell, wherein the locus is at least about 90% homologous to a sequence selected from SEQ ID NOs: 1-7 of WO 2019/126634 A2.

In some embodiments, a cell into which a polynucleotide of interest according to the present disclosure is to be introduced in accordance with the present disclosure is a ‘TI host cell’ according to any embodiment described in WO 2019/126634 A2, or is derived from a TI host cell according to any embodiment described in WO 2019/126634 A2. In some embodiments, a cell into which a polynucleotide of interest according to the present disclosure is to be introduced is a TI host cell according to any embodiment described in WO 2019/126634 A2, which has been further genetically-modified (e.g. to reduce or prevent expression of one or more genes).

In some embodiments, particularly embodiments in which the polynucleotide of interest is to be integrated into the genomic DNA of a cell via RMCE, the cell may further comprise/express the relevant recombinase, or nucleic acid encoding the relevant recombinase. For example, in some embodiments wherein the cell comprises genomic DNA comprising one or more target sequences for a given recombinase, the cell may further comprise/express the given recombinase, or nucleic acid encoding the given recombinase. By way of illustration, where the cell comprises genomic DNA comprising one or more lox sequences, the cell may further comprise/express Cre recombinase, or nucleic acid encoding Cre recombinase.

The cell may comprise the relevant recombinase/nucleic acid encoding the relevant recombinase as a consequence of having been engineered to comprise nucleic acid encoding the relevant recombinase. For example, nucleic acid encoding the relevant recombinase may have been introduced into the cell.

In some embodiments, the cell comprises extragenomic (i.e. non-genomic) nucleic acid encoding the relevant recombinase. In some embodiments, the cell comprises a vector comprising a nucleotide sequence encoding the relevant recombinase. In some embodiments, the genomic DNA of the cell comprises a nucleotide sequence encoding the relevant recombinase. In some embodiments, the cell transiently expresses the relevant recombinase.

Aspects and embodiments of the present disclosure relate to pluralities of cells according to the present disclosure.

Aspects and embodiments of the present disclosure relate to pluralities of cells comprising genomic DNA comprising a polynucleotide of interest (i.e. following integration of the polynucleotide of interest into the genomic DNA of such cells). In some embodiments, the plurality of cells comprises cells comprising genomic DNA comprising a polynucleotide of interest as a result of distinct integration events. For example, the plurality of cells may have been produced by a method in which a population of cells is contacted with a plurality of polynucleotides/vectors comprising a polynucleotide of interest under conditions providing for introduction of the polynucleotides/vectors into the cells and integration of the polynucleotide of interest into the genomic DNA of the cells, wherein each polynucleotide/vector of the plurality comprises a polynucleotide of interest having a unique identifier sequence.

Cells having a polynucleotide of interest integrated into their genomic DNA may be described as ‘stably’ comprising the polynucleotide of interest. Cells that stably comprise a given polynucleotide are distinguished from cells that transiently comprise the polynucleotide. In a cell that transiently comprises a given polynucleotide, the given polynucleotide may not be integrated into the genomic DNA of the cell, and the given polynucleotide may instead be extrachromosomal.

Cells having a polynucleotide of interest according to the present disclosure integrated into their genomic DNA may be described as ‘stably comprising’, the polynucleotide of interest. The progeny (i.e. daughter cells, following mitotic cell division) of a cell that stably comprises a given polynucleotide of interest also comprise the polynucleotide of interest. Cells that stably comprise a polynucleotide of interest are distinguished from cells that transiently express a polynucleotide of interest, in which the polynucleotide may instead be present as an extrachromosomal polynucleotide. Similarly, cells having a polynucleotide of interest according to the present disclosure integrated into their genomic DNA may be described as ‘stably expressing’, or having ‘stable expression of’, the polypeptide of interest encoded by the polynucleotide of interest. Cells that stably express a polypeptide of interest encoded by a polynucleotide of interest according to the present disclosure do so as a consequence of expression (by transcription and subsequent translation of mRNA) from the polynucleotide of interest, which is integrated into the genomic DNA of the cell. The progeny (i.e. daughter cells, following mitotic cell division) of a cell that stably expresses a polypeptide of interest also express the polypeptide of interest. Stable expression is distinguished from transient expression, which instead refers to expression of a polypeptide from a polynucleotide that is not integrated into the genomic DNA of the cell, e.g. an extrachromosomal polynucleotide.

The present disclosure provides a plurality of cells comprising genomic DNA comprising a polynucleotide of interest according to the present disclosure, wherein the genomic DNA of cells of the plurality comprise non-identical identifier sequences. Such pluralities of cells comprising cells having genomic DNA comprising non-identical identifier sequences may also be referred to libraries of cells, or polyclonal populations of cells.

In some embodiments, each cell of a plurality of cells comprising cells having genomic DNA comprising an identifier sequence has a unique identifier sequence (i.e. with respect to the identifier sequences of the genomic DNA of the other cells within the plurality).

In some embodiments, a plurality of cells comprising cells having genomic DNA comprising non-identical identifier sequences also comprises cells having the same identifier sequence, e.g. as a consequence of mitotic cell division of a cell comprising genomic DNA having a given identifier sequence. That is, in some embodiments, the plurality of cells comprises (i) cells having genomic DNA comprising non-identical identifier sequences (i.e. cells each comprising a polynucleotide of interest as a result of distinct integration events) and also comprises cells having genomic DNA comprising the same identifier sequence (i.e. cells that are the progeny of a cell comprising a polynucleotide of interest as a result of a given integration event).

The present disclosure also provides a plurality of cells in which each cell of the plurality comprises genomic DNA having the same identifier sequence. The cells of the plurality may the progeny of a cell comprising a polynucleotide of interest as a result of a given integration event, and the plurality of cells may have been produced as a consequence of mitotic cell division of a single given cell. Such pluralities of cells comprising cells having genomic DNA comprising the same identifier sequence may also be referred to as a monoclonal population of cells.

Producing Cells Comprising a Polynucleotide of Interest Integrated into their Genomic DNA

Aspects and embodiments of the present disclosure relate to producing cells comprising a polynucleotide of interest integrated into their genomic DNA.

The present disclosure provides methods for producing a cell comprising a polynucleotide of interest integrated into their genomic DNA, and the cells obtained or obtainable by such methods. Methods for producing cells comprising a given polynucleotide of interest integrated into their genomic DNA are well known to the skilled person, and generally comprise introducing polynucleotide(s)/vector(s) comprising the given polynucleotide of interest into the cells. Such methods may comprise nucleic acid transfer for permanent (i.e. stable) integration of the given polynucleotide of interest.

Any suitable genetic engineering platform may be used, and include gammaretroviral vectors, lentiviral vectors, adenovirus vectors, DNA transfection, transposon-based gene delivery and RNA transfection, for example as described in Maus et al., Annu Rev Immunol (2014) 32:189-225, hereby incorporated by reference in its entirety. Methods also include those described e.g. in Wang and Rivière Mol Ther Oncolytics. (2016) 3:16015, which is hereby incorporated by reference in its entirety. Suitable methods for introducing polynucleotide(s)/vector(s) into cells include transduction, transfection and electroporation.

Cells comprising a polynucleotide of interest according to the present disclosure integrated into their genomic DNA may be produced by a method comprising: contacting cells with polynucleotide(s)/vector(s) comprising a polynucleotide of interest according to the present disclosure under conditions suitable for introducing the polynucleotide(s)/vector(s) into the cells. The person skilled in the art is aware of suitable conditions for effective introduction of a polynucleotide/vector into a cell in culture.

In some embodiments, the method further comprises subjecting cells into which a polynucleotide/vector comprising a polynucleotide of interest has been introduced to conditions suitable for the integration of the polynucleotide into the genomic DNA of the cell.

In embodiments wherein the polynucleotide/vector comprising a polynucleotide of interest and/or the cell into which the polynucleotide/vector is introduced comprise nucleotide sequence(s) providing for integration of the polynucleotide of interest into the genomic DNA of the cell via RMCE, the method may comprise culturing the cells under conditions suitable for RMCE to occur. For example, the methods may comprise culturing the cells under conditions suitable the expression and/or recombinase activity of the relevant recombinase. By way of illustration, in embodiments wherein the genomic DNA of the cell comprises one or more lox sequences, the polynucleotide/vector comprising a polynucleotide of interest comprises lox sequences flanking the nucleotide sequence of a polynucleotide of interest, and the cell further comprises a nucleotide sequence encoding Cre recombinase, the method may comprise culturing the cells under conditions suitable for expression of Cre recombinase from the nucleotide sequence encoding Cre recombinase, and/or Cre recombinase-mediated integration of the polynucleotide of interest into the genomic DNA of the cell.

In some embodiments, the methods comprise subjecting cells that have been subjected to conditions suitable for introducing a polynucleotide/vector comprising a polynucleotide of interest into the cell and subsequent integration of the polynucleotide of interest into the genomic DNA of the cell to selection for cells having integrated the polynucleotide of interest into their genomic DNA.

In some embodiments, the methods comprise culturing the cells in the presence of a cytotoxic compound to which the cell is susceptible in the absence of integration of the polynucleotide of interest, and to which the cells having integrated the polynucleotide of interest display resistance. In embodiments wherein the polynucleotide of interest comprises a nucleotide sequence encoding a selectable marker, the methods may comprise culturing the cells in the presence of the cytotoxic compound to which the selectable marker confers resistance. By way of illustration, in embodiments in which the polynucleotide of interest comprises an antibiotic resistance gene (e.g. a nucleotide sequence encoding puromycin N-acetyltransferase), the methods may comprise culturing the cells in the presence of the relevant antibiotic (e.g. puromycin).

### Analyzing Identifier Sequences

Aspects and embodiments of the present disclosure involve analyzing one or more cells to determine the identity of an identifier sequence they comprise. That is, aspects and embodiments comprise analyzing nucleic acid of a cell having integrated a polynucleotide of interest into its genomic DNA to determine the identity of the identifier sequence.

The analysis may comprise determining the structure of the identifier sequence. In some embodiments, the analysis comprises determining the nucleotide sequence of an identifier sequence. The methods may comprise extracting genomic DNA from a cell having integrated a polynucleotide of interest into its genomic DNA, and subsequently analyzing the nucleotide sequence of the identifier sequence integrated into the genomic DNA of the cell.

The nucleotide sequence of a polynucleotide may be determined by any suitable techniques, which are well known to the skilled person, and include e.g. Sanger sequencing. In preferred embodiments, next-generation sequencing (NGS) technology may be employed to determine the nucleotide sequence of an identifier sequence according to the present disclosure. The analysis may employ oligonucleotide primers for the amplification of a nucleotide sequence comprising or consisting of the identifier sequence, e.g. by polymerase chain reaction.

### Producing Populations of Cells

Aspects and embodiments of the present disclosure involve culturing cells (e.g. in vitro) to increase their number. Such aspects and embodiments may comprise culturing a cell under conditions so as to generate a population of such cells, e.g. as a consequence of cell division.

Such aspects and embodiments may comprise culturing plurality of cells under conditions suitable to expand their number. That is, in some aspects and embodiments, a population of cells may be cultured under conditions suitable for expansion of the population of cells.

Suitable culture conditions will be apparent to a person skilled in the art. Culture conditions for the culture of mammalian cells are described e.g. in Birch and Racher, Adv Drug Deliv Rev. (2006) 58 (5-6): 671-85 and Li et al., MAbs (2010) 2 (5): 466-477, both of which are hereby incorporated by reference in their entirety. Suitable culture conditions include conditions suitable for the maintenance of cells of the CHO cells in in vitro culture.

The cells are cultured in cell culture medium comprising amino acids, vitamins, inorganic salts and sugars. In some embodiments, the cell culture medium comprises amino acids selected from: L-alanine, L-arginine, L-asparagine, L-aspartic acid, L-cystine/L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. In some embodiments, the cell culture medium comprises vitamins and/or vitaminoids selected from: D-biotin, choline chloride, D-calcium pantothenate, folic acid, myo-inositol, niacinamide, pyridoxine hydrochloride, riboflavin, thiamine hydrochloride, and vitamin B12. In some embodiments, the cell culture medium comprises further components selected from: calcium chloride, hypoxanthine, ferric nitrate, linoleic acid, putrescine hydrochloride, pyruvic acid, magnesium sulfate, potassium chloride, sodium bicarbonate, sodium chloride, sodium phosphate monobasic, thioctic acid and thymidine. In some embodiments, the cell culture medium comprises D-glucose.

In some embodiments, the cell culture medium is a cell culture medium suitable for the culture of mammalian cells. Such cell culture media include Roswell Park Memorial Institute (RPMI) 1640 medium, Dulbecco's Modified Eagle Medium (DMEM), F-12 medium, DMEM/F12, CD-CHO medium and PowerCHO medium.

In preferred embodiments, the cell culture medium is suitable cell culture medium for the culture of cells for the production of molecules to be employed in therapy in humans. Such culture medium includes e.g. EX-CELL Advanced CHO Fed-Batch Medium.

It will be appreciated that the cells are cultured under suitable environmental conditions. The cells may be cultured at 28 °C to 38 °C, e.g. at one of about 28 °C, about 28.5 °C, about 29 °C, about 29.5 °C, about 30 °C, about 30.5 °C, about 31 °C, about 31.5 °C, about 32 °C, about 32.5 °C, about 33 °C, about 33.5 °C, about 34 °C, about 34.5 °C, about 35 °C, about 35.5 °C, about 36 °C, about 36.5 °C, about 37 °C, about 37.5 °C or about 38 °C. The cells may be cultured in 4% to 10% CO₂, e.g. 5% to 8% CO2. The cells may be cultured e.g. at ≥90% humidity, e.g. about 95% humidity. The cells may be cultured without agitation, or with agitation. Agitation may be at 75 rpm to 175 rpm, e.g. 90 rpm to 130 rpm, e.g. about 110 rpm. The pH of the cell culture may be between 6.8 to 7.4, e.g. one of about 6.8, 6.9, 7.0, 7.1, 7.2, 7.3 or 7.4. In some embodiments, the pH of the cell culture is about 7.0. In some embodiments, the pH of the cell culture is about 7.2.

Cell culture may be performed in a bioreactor provided with an appropriate supply of nutrients, air/oxygen and/or growth factors. Bioreactors may monitor and control environmental conditions such as pH, oxygen, flow rates into and out of, and agitation within the vessel such that optimum conditions are provided for the cells being cultured.

The culture may be a continuous culture, with a continuous flow of cell culture medium into, and a continuous flow of cultured cells from, the cell culture vessel. In some embodiments, the culture may be a batch culture, employing a closed system and a finite amount of cell culture medium. In some embodiments, the culture may be a fed-batch culture, in which cell culture medium is supplied to the cell culture vessel during the culture, but wherein unlike a continuous culture, material is not removed from the cell culture vessel during the course of cell culture.

In some embodiments, the methods comprise adding one or more components to the cell culture during the period of culture. In some embodiments, the methods comprise adding cell culture medium to the cell culture during the period of culture. In some embodiments, the methods comprise adding nutrients to the cell culture during the period of culture. In some embodiments, the methods comprise adding amino acids to the cell culture during the period of culture.

Aspects and embodiments of the present disclosure contemplate selecting certain cells for subsequent culture and/or expansion over others.

In some aspects and embodiments, a cell or a plurality of cells is/are selected for subsequent culture and/or expansion on the basis of the identity of an identifier sequence comprised in their genomic DNA. In some embodiments, methods according to the present disclosure comprise separating/sorting/partitioning cells on the basis of the identity of their identifier sequence.

In some embodiments, a cell having genomic DNA comprising a polynucleotide of interest having a given identifier sequence is separated/sorted/partitioned form another cell comprising having genomic DNA comprising a polynucleotide of interest having an identifier sequence that is different to the given identifier sequence.

### Applications of the Technology

The articles (e.g. the polynucleotides, vectors, cells) and the methods of the present disclosure find use in a variety of applications, particularly in the context of biomolecule production.

The use of unique identifier sequences in the polynucleotides of interest has the result that cells comprising a polynucleotide of interest as a consequence of targeted integration events can be identified. Where cells of the plurality comprise non-identical identifier sequences, they must comprise polynucleotides of interest as a consequence of multiple, different targeted integration events. Conversely, where two or more cells of the plurality comprise the same identifier sequence, they must comprise a polynucleotide of interest as a consequence of the same targeted integration event, and must therefore be the progeny of the cell in which targeted integration of the relevant polynucleotide of interest took place.

Cells derived from the same parent cell typically have similar characteristics to one another, e.g. in terms of growth characteristics (e.g. doubling time), metabolic profile, optimal culture conditions, productivity (e.g. in terms of production of a biomolecule, e.g. expression of a polypeptide of interest encoded by a polynucleotide of interest according to the present disclosure), etc. Conversely, cells derived from different parent cells sometimes have different characteristics.

In some instances, it can be desirable to obtain/maintain a monoclonal population of cells and/or to reduce the diversity of a polyclonal population of cells, e.g. for predictability and in order to maximise consistency of performance of the cells in culture (e.g. in terms of growth characteristics, culture requirements, productivity, etc.). In other instances, it might be desirable to obtain/maintain a polyclonal population of cells and/or to increase the diversity of a polyclonal population of cells, e.g. to maintain sufficient diversity in order that cells having a desirable profile of characteristics (e.g. in terms of growth characteristics, culture requirements, productivity, etc.) can be identified and subsequently selected.

The articles and methods are useful for obtaining/producing monoclonal populations of cells having a polynucleotide of interest integrated into their genomic DNA. Through analysis of the identifier sequences (i.e. identifier sequences of integrated polynucleotides of interest) of cells obtained by (a) contacting a population of cells with a plurality of vectors under conditions suitable for introducing the vectors into the cells, wherein each vector of the plurality of vectors comprises a nucleotide sequence providing for targeted integration of a polynucleotide of interest comprising an identifier sequence into the genomic DNA of a cell into which the vector has been introduced, and wherein each vector of the plurality of vectors comprises a polynucleotide of interest having a unique identifier sequence; and (b) subjecting the cells obtained after step (a) to selection for cells having integrated a polynucleotide of interest into their genomic DNA, it is possible to identify a cell or cells arising from a given integration event. Such cell(s) can then be selected and separated/sorted/partitioned from cells having different identifier sequences, thereby obtaining a clone/monoclonal population of cells. The cell(s) may thereafter be subsequently cultured (i.e. in isolation from cells having different identifier sequences) to increase their number/expand the population.

The articles and methods of the present disclosure are also useful for obtaining/producing libraries of genetically non-identical cells (i.e. a polyclonal population of cells) having a polynucleotide of interest integrated into their genomic DNA. Through analysis of the identifier sequences (i.e. identifier sequences of integrated polynucleotides of interest) of cells obtained by (a) contacting a population of cells with a plurality of vectors under conditions suitable for introducing the vectors into the cells, wherein each vector of the plurality of vectors comprises a nucleotide sequence providing for targeted integration of a polynucleotide of interest comprising an identifier sequence into the genomic DNA of a cell into which the vector has been introduced, and wherein each vector of the plurality of vectors comprises a polynucleotide of interest having a unique identifier sequence; and (b) subjecting the cells obtained after step (a) to selection for cells having integrated a polynucleotide of interest into their genomic DNA, it is possible to identify cells arising from different integration events. Cells arising from different integration events can then be selected, thereby obtaining a library/polyclonal population of cells. The cell(s) may thereafter be subsequently cultured to increase their number/expand the population.

The articles and methods of the present disclosure also find use in monitoring and/or managing (e.g. decreasing or increasing) the diversity of pluralities of cells comprising polynucleotides of interest according to the present disclosure, through analysis of cells obtained by (a) contacting a population of cells with a plurality of vectors under conditions suitable for introducing the vectors into the cells, wherein each vector of the plurality of vectors comprises a nucleotide sequence providing for targeted integration of a polynucleotide of interest comprising an identifier sequence into the genomic DNA of a cell into which the vector has been introduced, and wherein each vector of the plurality of vectors comprises a polynucleotide of interest having a unique identifier sequence; and (b) subjecting the cells obtained after step (a) to selection for cells having integrated a polynucleotide of interest into their genomic DNA, to determine the identity of their identifier sequences. In some embodiments, the methods further comprise determining the proportion of cells in the population having integrated a given identifier sequence.

Evaluation of the diversity of pluralities of cells comprising polynucleotides of interest according to the present disclosure also enables the purity of a putative monoclonal population of cells to be monitored/evaluated/confirmed. Accordingly, present disclosure provides methods and uses relating to monitoring/evaluating/confirming the monoclonality of a putative monoclonal population of cells. Unintended contamination of putative monoclonal cell cultures with other cells can sometimes occur. Where such analysis detects only a single identifier sequence among cells of the population, the purity/monoclonality of the population is confirmed. Where such analysis detects one or more additional identifier sequences among cells of the population, the population is determined to be contaminated or not monoclonal. In such instances, a cell or cells arising from a given integration event (e.g. having the expected identifier sequence) may subsequently be selected and separated/sorted/partitioned from cells having different identifier sequences, thereby obtaining a clone/monoclonal population of cells, and the cell(s) may thereafter be subsequently cultured (i.e. in isolation from cells having different identifier sequences) to increase their number/expand the population.

It will be appreciated that in some aspects and embodiments in accordance with the present disclosure, a method for evaluating the diversity of a population of cells having a polynucleotide of interest integrated into their genomic DNA is a method for monitoring/evaluating/confirming the monoclonality of a putative monoclonal population of cells. It will also be appreciated that in some aspects and embodiments, a method for confirming the identity of a population of cells having a polynucleotide of interest integrated into their genomic DNA may be a method for monitoring/evaluating/confirming the monoclonality of a putative monoclonal population of cells.

For example, such evaluation also enables the identity of a monoclonal population of cells to be evaluated/confirmed. Unintended mixing-up of cell cultures can sometimes occur. Where such analysis detects the expected identifier sequence among cells of the population, the identity of the population is confirmed.

In some instances, it may be desirable to preserve/maintain or increase the genetic diversity of a plurality of genetically non-identical cells (i.e. a polyclonal population of cells) having a polynucleotide of interest integrated into their genomic DNA. Where a plurality of genetically non-identical cells is cultured, over time one or more clones of the population may expand to become the dominant clone(s), constituting a larger proportion of the polyclonal population than other clones. This can in turn result in a loss of genetic diversity for the polyclonal population of cells, as other clones are out-competed. Such a reduction in genetic diversity of the polyclonal population might not be desirable, as it might inhibit/prevent the identification of cells having a desirable profile of characteristics (e.g. in terms of growth characteristics, culture requirements, productivity, etc.). Accordingly, in such instances, cells arising from different integration events can be selected from the population, and subsequently cultured to obtain a polyclonal population of cells. In some embodiments, cell(s) from clones that were dominant clones in the population prior to such selection might not be selected for subsequent culture.

Evaluation of the diversity of pluralities of cells comprising polynucleotides of interest according to the present disclosure also enables the diversity and/or population dynamics of a plurality of genetically non-identical cells (i.e. a polyclonal population of cells) having a polynucleotide of interest integrated into their genomic DNA to be monitored over time. Repeated analysis of cells maintained in culture to determine the identity of their identifier sequences at different time points enables detection of changes in the numbers/proportions of cells having given identifier sequence(s) over time.

Evaluation in accordance with the preceding paragraph may be employed in an iterative process for optimising the culture conditions of polyclonal populations of cells having desired diversity. In such instances, the diversity of a polyclonal population of cells produced following culture under given culture conditions may be determined, and compared with the diversity of a polyclonal population of cells produced following culture under different conditions. Conditions for culture of a polyclonal population of cells for optimal diversity can thereby be inferred.

The articles and methods of the present disclosure also find use in evaluating the performance of and/or optimising methods for the production of populations of cells having a polynucleotide of interest integrated into their genomic DNA. Through analysis of the identifier sequences (i.e. identifier sequences of integrated polynucleotides of interest) of cells obtained by (a) contacting a population of cells with a plurality of vectors under conditions suitable for introducing the vectors into the cells, wherein each vector of the plurality of vectors comprises a nucleotide sequence providing for targeted integration of a polynucleotide of interest comprising an identifier sequence into the genomic DNA of a cell into which the vector has been introduced, and wherein each vector of the plurality of vectors comprises a polynucleotide of interest having a unique identifier sequence; and (b) subjecting the cells obtained after step (a) to selection for cells having integrated a polynucleotide of interest into their genomic DNA, it is possible to identify conditions for method steps (a) and/or (b) providing for the production of more or less diverse polyclonal populations of cells. In particular, it might be desirable to determine conditions for methods step (a) and/or (b) providing for the production of highly-diverse polyclonal populations of cells, in order that cells having a desirable profile of characteristics (e.g. in terms of growth characteristics, culture requirements, productivity) can be identified and subsequently selected.

Evaluation in accordance with the preceding paragraph may be employed in an iterative process for optimising method steps according to (a) and/or (b), for the production of polyclonal populations of cells with desired diversity. In such instances, the diversity of a polyclonal population of cells produced by a given set of conditions for method steps (a) and/or (b) may be determined, and compared with the diversity of a polyclonal population of cells produced by different conditions for method steps (a) and/or (b). Conditions for method steps (a) and/or (b) providing for optimal diversity in the polyclonal population of cells obtained can thereby be inferred.

### Aspects and Embodiments Relating to Cells Integrating Multiple Polynucleotides of Interest

As explained hereinabove, aspects and embodiments of the present disclosure relate to methods and uses wherein cells are modified such that their genomic DNA comprises a plurality of (e.g. 2, 3, 4, 5, 6, 7, 8 or more) integrated polynucleotides of interest. In accordance with such aspects and embodiments, each polynucleotide of interest of the plurality preferably comprises a different nucleotide sequence of interest (e.g. encoding non-identical polypeptides/nucleic acids of interest).

In the following aspects and embodiments, reference is made to ‘two or more polynucleotides of interest’. It will be appreciated that this encompasses embodiments relating e.g. to three, four, five, six, seven, eight or more polynucleotides of interest.

It will similarly be appreciated that ‘a first polynucleotide of interest comprising an identifier sequence and a second polynucleotide of interest comprising an identifier sequence’ encompasses e.g. a first polynucleotide of interest comprising an identifier sequence, a second polynucleotide of interest comprising an identifier sequence and a third polynucleotide of interest comprising an identifier sequence; or a first polynucleotide of interest comprising an identifier sequence, a second polynucleotide of interest comprising an identifier sequence, a third polynucleotide of interest comprising an identifier sequence, and a fourth polynucleotide of interest comprising an identifier sequence; or a first polynucleotide of interest comprising an identifier sequence, a second polynucleotide of interest comprising an identifier sequence, a third polynucleotide of interest comprising an identifier sequence, a fourth polynucleotide of interest comprising an identifier sequence and a fifth polynucleotide of interest comprising an identifier sequence; etc. It will be appreciated that the identifier sequences of different polynucleotides of interest are non-identical (that is, the identifier sequence of a first polynucleotide of interest comprising an identifier sequence and the identifier sequence of a second polynucleotide of interest comprising an identifier sequence are not the same). Indeed, the identifier sequences of individual copies of a given polynucleotide of interest are non-identical. In accordance with such embodiments, in each vector comprising the relevant polynucleotide of interest (e.g. the third polynucleotide of interest, fourth polynucleotide, etc.), the identifier sequence is unique.

Accordingly, the present disclosure provides a method for evaluating the diversity of a library of genetically non-identical cells having two or more polynucleotides of interest integrated into their genomic DNA, comprising analyzing cells obtained by:
(a) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique; and
(b) subjecting the cells obtained after step (a) to selection for cells having integrated a first polynucleotide of interest and a second polynucleotide of interest into their genomic DNA;
to determine the identity of the identifier sequences integrated into their genomic DNA.

In some embodiments, the method further comprises determining the proportion of cells in the population having integrated a given identifier sequence.

The present disclosure provides a method for obtaining a library of genetically non-identical cells having two or more polynucleotides of interest integrated into their genomic DNA, comprising:
(i) analyzing cells obtained by:
   (a) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique; and
   (b) subjecting the cells obtained after step (a) to selection for cells having integrated a first polynucleotide of interest and a second polynucleotide of interest into their genomic DNA;
to determine the identity of the identifier sequences integrated into their genomic DNA; and
(ii) selecting cells determined in step (i) to have profiles of identifier sequences that are non-identical for subsequent expansion.

In accordance with the preceding paragraph, it will be appreciated that a profile of identifier sequences (i.e. for a given cell having integrated a first polynucleotide of interest and a second polynucleotide of interest into their genomic DNA) refers to the combination of identifier sequences from the first polynucleotide of interest and the second polynucleotide of interest. It will also be appreciated that the identifier sequence from the first polynucleotide of interest is different to the identifier sequence from the second polynucleotide of interest.

By way of illustration, a first cell may have integrated a first polynucleotide of interest comprising identifier sequence A, and a second polynucleotide of interest comprising identifier sequence X. A second cell having a non-identical profile of identifier sequences to the first cell may comprise a first polynucleotide of interest comprising an identifier sequence other than A (e.g. may comprise a first polynucleotide of interest comprising e.g. identifier sequence B or C), and/or may comprise a second polynucleotide of interest comprising an identifier sequence other than X (e.g. may comprise a second polynucleotide of interest comprising e.g. identifier sequence Y or Z). Conversely, a second cell having an identical profile of identifier sequences to the first cell comprises a first polynucleotide of interest comprising identifier sequence A, and a second polynucleotide of interest comprising identifier sequence X.

The present disclosure provides a method for obtaining a library of genetically non-identical cells having two or more polynucleotides of interest integrated into their genomic DNA, comprising:
(i) analyzing cells obtained by:
   (a) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique; and
   (b) subjecting the cells obtained after step (a) to selection for cells having integrated a first polynucleotide of interest and a second polynucleotide of interest into their genomic DNA;
to determine the identity of the identifier sequences integrated into their genomic DNA; and
(ii) selecting a single cell, or selecting a plurality of cells determined in step (i) to have an identical profile of identifier sequences, for subsequent expansion.

The present disclosure also provides a method for evaluating the diversity of a population of cells having two or more polynucleotides of interest integrated into their genomic DNA, comprising analyzing cells obtained by:
(a) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique;
(b) subjecting the cells obtained after step (a) to selection for cells having integrated a first polynucleotide of interest and a second polynucleotide of interest into their genomic DNA;
(c) analyzing cells obtained after step (b) to determine the identity of the identifier sequences integrated into their genomic DNA; and
(d) selecting cells determined in step (c) to have profiles of identifier sequences that are non-identical for subsequent expansion;
to determine the identity of the identifier sequences integrated into their genomic DNA.

The present disclosure also provides a method for confirming the identity of a population of cells having two or more polynucleotides of interest integrated into their genomic DNA, comprising analyzing cells obtained by:
(a) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique;
(b) subjecting the cells obtained after step (a) to selection for cells having integrated a first polynucleotide of interest and a second polynucleotide of interest into their genomic DNA;
(c) analyzing cells obtained after step (b) to determine the identity of the identifier sequences integrated into their genomic DNA; and
(d) selecting a single cell, or selecting a plurality of cells determined in step (c) to have an identical profile of identifier sequences, for subsequent expansion;
to determine the identity of the identifier sequences integrated into their genomic DNA.

The present disclosure also provides a method for evaluating the diversity of a library of genetically non-identical cells having two or more polynucleotides of interest integrated into their genomic DNA, comprising:
(i) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique;
(ii) subjecting the cells obtained after step (a) to selection for cells having integrated a first polynucleotide of interest and a second polynucleotide of interest into their genomic DNA; and
(iii) analyzing cells obtained after step (ii) to determine the identity of the identifier sequences integrated into their genomic DNA.

In some embodiments, the method further comprises determining the proportion of cells in the population obtained after step (ii) having integrated a given identifier sequence.

The present disclosure also provides a method for obtaining a library of genetically non-identical cells having two or more polynucleotides of interest integrated into their genomic DNA, comprising:
(i) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique;
(ii) subjecting the cells obtained after step (a) to selection for cells having integrated a first polynucleotide of interest and a second polynucleotide of interest into their genomic DNA; and
(iii) analyzing cells obtained after step (ii) to determine the identity of the identifier sequences integrated into their genomic DNA; and
(iv) selecting cells determined in step (iii) to have profiles of identifier sequences that are non-identical for subsequent expansion.

The present disclosure also provides a method for obtaining a monoclonal population of cells having two or more polynucleotides of interest integrated into their genomic DNA, comprising:
(i) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique;
(ii) subjecting the cells obtained after step (a) to selection for cells having integrated a first polynucleotide of interest and a second polynucleotide of interest into their genomic DNA; and
(iii) analyzing cells obtained after step (ii) to determine the identity of the identifier sequences integrated into their genomic DNA; and
(iv) selecting a single cell, or selecting a plurality of cells determined in step (i) to have an identical profile of identifier sequences, for subsequent expansion.

The present disclosure also provides a method for evaluating the diversity of a population of cells having two or more polynucleotides of interest integrated into their genomic DNA, comprising:
(i) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique;
(ii) subjecting the cells obtained after step (a) to selection for cells having integrated a first polynucleotide of interest and a second polynucleotide of interest into their genomic DNA; and
(iii) analyzing cells obtained after step (ii) to determine the identity of the identifier sequences integrated into their genomic DNA; and
(iv) selecting a single cell, or selecting a plurality of cells determined in step (i) to have an identical profile of identifier sequences, for subsequent expansion; and
(v) analyzing cells obtained after step (iv) to determine the identity of the identifier sequences integrated into their genomic DNA.

In some embodiments, the method for evaluating the diversity of a population of cells having two or more polynucleotides of interest integrated into their genomic DNA is a method for monitoring or evaluating the monoclonality of a putative monoclonal population of cells having two or more polynucleotides of interest integrated into their genomic DNA.

The present disclosure also provides a method for confirming the identity of a population of cells having two or more polynucleotides of interest integrated into their genomic DNA, comprising:
(i) contacting a population of cells with a plurality of vectors comprising nucleotide sequences providing for targeted integration of at least a first polynucleotide of interest comprising an identifier sequence and a second polynucleotide of interest comprising an identifier sequence into the genomic DNA of a cell, under conditions suitable for introducing the vectors into the cells; wherein in each vector of the plurality of vectors that comprises a first polynucleotide of interest, the identifier sequence is unique; and wherein in each vector comprising a second polynucleotide of interest, the identifier sequence is unique;
(ii) subjecting the cells obtained after step (a) to selection for cells having integrated a first polynucleotide of interest and a second polynucleotide of interest into their genomic DNA; and
(iii) analyzing cells obtained after step (ii) to determine the identity of the identifier sequences integrated into their genomic DNA; and
(iv) selecting a single cell, or selecting a plurality of cells determined in step (i) to have an identical profile of identifier sequences, for subsequent expansion; and
(v) analyzing cells obtained after step (iv) to determine the identity of the identifier sequences integrated into their genomic DNA.

In some embodiments, the method for confirming the identity of a population of cells having two or more polynucleotides of interest integrated into their genomic DNA is a method for confirming the monoclonality of a putative monoclonal population of cells having two or more polynucleotides of interest integrated into their genomic DNA.

The present disclosure also provides the use of a plurality of vectors comprising nucleotide sequences providing for targeted integration of at least a first polynucleotide of interest comprising an identifier sequence and a second polynucleotide of interest comprising an identifier sequence into the genomic DNA of a cell in a method for evaluating the diversity of a library of genetically non-identical cells having two or more polynucleotides of interest integrated into their genomic DNA, wherein in each vector of the plurality of vectors that comprises a first polynucleotide of interest, the identifier sequence is unique; and wherein in each vector comprising a second polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a first polynucleotide of interest and a second polynucleotide of interest into their genomic DNA; and
(iii) analyzing cells obtained after step (ii) to determine the identity of the identifier sequences integrated into their genomic DNA.

In some embodiments, the method further comprises determining the proportion of cells in the population obtained after step (ii) having integrated a given identifier sequence.

The present disclosure also provides the use of a plurality of vectors comprising nucleotide sequences providing for targeted integration of at least a first polynucleotide of interest comprising an identifier sequence and a second polynucleotide of interest comprising an identifier sequence into the genomic DNA of a cell in a method for obtaining a clonal population of cells having two or more polynucleotides of interest integrated into their genomic DNA, wherein in each vector of the plurality of vectors that comprises a first polynucleotide of interest, the identifier sequence is unique; and wherein in each vector comprising a second polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a first polynucleotide of interest and a second polynucleotide of interest into their genomic DNA;
(iii) analyzing cells obtained after step (ii) to determine the identity of the identifier sequences integrated into their genomic DNA.
(iv) selecting cells determined in step (iii) to have profiles of identifier sequences that are non-identical for subsequent expansion.

The present disclosure also provides the use of a plurality of vectors comprising nucleotide sequences providing for targeted integration of at least a first polynucleotide of interest comprising an identifier sequence and a second polynucleotide of interest comprising an identifier sequence into the genomic DNA of a cell in a method for obtaining a library of genetically non-identical cells having two or more polynucleotides of interest integrated into their genomic DNA, wherein in each vector of the plurality of vectors that comprises a first polynucleotide of interest, the identifier sequence is unique; and wherein in each vector comprising a second polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a first polynucleotide of interest and a second polynucleotide of interest into their genomic DNA;
(iii) analyzing cells obtained after step (ii) to determine the identity of the identifier sequences integrated into their genomic DNA;
(iv) selecting a single cell, or selecting a plurality of cells determined in step (iii) to have an identical profile of identifier sequences, for subsequent expansion.

The present disclosure also provides the use of a plurality of vectors comprising nucleotide sequences providing for targeted integration of at least a first polynucleotide of interest comprising an identifier sequence and a second polynucleotide of interest comprising an identifier sequence into the genomic DNA of a cell in a method for evaluating the diversity of a population of cells having a polynucleotide of interest integrated into their genomic DNA, wherein in each vector of the plurality of vectors that comprises a first polynucleotide of interest, the identifier sequence is unique; and wherein in each vector comprising a second polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a first polynucleotide of interest and a second polynucleotide of interest into their genomic DNA;
(iii) analyzing cells obtained after step (ii) to determine the identity of the identifier sequences integrated into their genomic DNA;
(iv) selecting a single cell, or selecting a plurality of cells determined in step (iii) to have an identical profile of identifier sequences, for subsequent expansion; and
(v) analyzing cells obtained after step (iv) to determine the identity of the identifier sequences integrated into their genomic DNA.

In some embodiments, the method for evaluating the diversity of a population of cells having two or more polynucleotides of interest integrated into their genomic DNA is a method for monitoring or evaluating the monoclonality of a putative monoclonal population of cells having two or more polynucleotides of interest integrated into their genomic DNA.

The present disclosure also provides the use of a plurality of vectors comprising nucleotide sequences providing for targeted integration of at least a first polynucleotide of interest comprising an identifier sequence and a second polynucleotide of interest comprising an identifier sequence into the genomic DNA of a cell in a method for confirming the identity of a population of cells having a polynucleotide of interest integrated into their genomic DNA, wherein in each vector of the plurality of vectors that comprises a first polynucleotide of interest, the identifier sequence is unique; and wherein in each vector comprising a second polynucleotide of interest, the identifier sequence is unique, and wherein the method comprises:
(i) contacting a population of cells with the plurality of vectors under conditions suitable for introducing the vectors into the cells;
(ii) subjecting the cells obtained after step (i) to selection for cells having integrated a first polynucleotide of interest and a second polynucleotide of interest into their genomic DNA;
(iii) analyzing cells obtained after step (ii) to determine the identity of the identifier sequences integrated into their genomic DNA;
(iv) selecting a single cell, or selecting a plurality of cells determined in step (iii) to have an identical profile of identifier sequences, for subsequent expansion; and
(v) analyzing cells obtained after step (iv) to determine the identity of the identifier sequences integrated into their genomic DNA.

In some embodiments, the method for confirming the identity of a population of cells having two or more polynucleotides of interest integrated into their genomic DNA is a method for confirming the monoclonality of a putative monoclonal population of cells having two or more polynucleotides of interest integrated into their genomic DNA.

In some embodiments, prior to integration of the two or more polynucleotides of interest, the cells comprise genomic DNA having single-copy landing pads providing for targeted integration of the two or more polynucleotides of interest.

In some embodiments, the two or more polynucleotides of interest comprise a nucleotide sequence encoding one or more polypeptides of interest.

In some embodiments, the one or more polypeptides of interest are each independently selected from the group consisting of: an antigen-binding polypeptide, an aptamer, a constituent polypeptide of an antigen-binding polypeptide complex, an antibody/antigen-binding fragment or derivative thereof, a constituent polypeptide of an antibody/antigen-binding fragment or derivative thereof, an Fc fusion polypeptide, an anticoagulant, a blood factor, a bone morphogenetic polypeptide, a decoy receptor for a ligand, a decoy ligand for a receptor, an enzyme, an enzyme co-factor, a growth factor, a hormone, an interferon, an interleukin, a thrombolytic, a transcription factor, an epigenetic modifier, a constituent polypeptide of a site-specific nuclease nucleic acid editing system, a constituent polypeptide of a ribonucleoprotein, a viral polypeptide, or a polypeptide useful for the production of a biomolecule.

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

As used herein, an amino acid sequence, or a region of a polypeptide which ‘correponds’ to a specified reference amino acid sequence or region of a polypeptide has at least 60%, e.g. one of at least ≥65%, ≥70%, ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the amino acid sequence/polypeptide/region. An amino acid sequence/region/position of a polypeptide/amino acid sequence which ‘corresponds’ to a specified reference amino acid sequence/region/position of a polypeptide/amino acid sequence can be identified by sequence alignment of the subject sequence to the reference sequence, e.g. using sequence alignment software such as ClustalOmega (Soding, J. 2005, Bioinformatics 21, 951-960).

Where an amino acid (other than glycine) is referred to herein, both L and D enantiomers of the relevant amino acid are expressly contemplated. In some embodiments, except where specifically indicated otherwise, reference to an amino acid herein refers particularly to the L enantiomer, this being the form of the amino acid as it occurs in nature.

It must be noted that, as used in the specification and the appended claims, the singular forms ‘a’, ‘an’, and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about’, it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

Values may be expressed herein as ‘about’ a particular value. Similarly, ranges may be expressed herein as from ‘about’ a particular value, and/or to ‘about’ another particular value. The term ‘about’ in relation to a numerical value is optional, and means for example +/−10%. By way of illustration, reference e.g. to ‘about 10%’ is to be construed as 9% to 11%. In instances herein where ‘about’ is recited, the value it precedes is also specifically contemplated. By way of illustration, reference e.g. to ‘about 10%’ also specifically contemplates 10%.

### BRIEF DESCRIPTION OF THE FIGURES

Embodiments and experiments illustrating the principles of the present disclosure will now be discussed with reference to the accompanying figures.

FIGS. 1A-1E. Targeted barcode integration into stable CHO producer cells. (FIG. 1A) Schematic of a targeted integration locus in CHO cells containing front and back plasmids and barcode library adjacent to the L2 lox site. (FIG. 1B) Experimental timeline for evaluation of stable pool composition during cell line development. (FIG. 1C) Schematic of the cell pool selection and recovery process. (FIG. 1D) Left: Cell pool recovery kinetics, shown by % cell viability; and Right: barcode fraction pre- and post-selection for three antibody molecules of increasing complexity: M1 (top panels), M2 (middle panels) and M3 (bottom panels). (FIG. 1E) Pool composition over 80 days measured by barcode fraction in post-recovery CHO cell pools expressing three antibody molecules of increasing complexity: M1 (top panels), M2 (middle panels) and M3 (bottom panels).

FIGS. 2A-2D. Validation of plasmid library. (FIG. 2A) Diversity of Back plasmid barcode libraries assessed by amplicon deep sequencing shows uniform barcode representation. (FIG. 2B) (SEQ ID NOs: 4 to 11) Homogenous nucleotide probability at each position of the N15 barcode region. (FIG. 2C) Estimation of lower bound of total barcode diversity within each library based on observed counts using the Chao1 capture-recapture estimator. (FIG. 2D) Estimation of labeling capacity for each library by coincidental barcode collisions. Mean values for 100 replicate simulations (resampling).

FIGS. 3A-3E. Barcoding allows for discrimination between cell-clones originating from the same RMCE event or different RMCE events. (FIG. 3A) Experimental outline to evaluate the cellular production performance of clonal cells which originated from different RMCE events. (FIG. 3B) (SEQ ID NOs: 12 to 22) Clustering of cell-clones sharing the same barcode is based partially on antibody chain expression. (FIGS. 3C-3E) Absolute differences in (FIG. 3C) product quality parameters, (FIG. 3D) metabolite concentrations and (FIG. 3E) cellular features between unique barcodes as compared to barcodes with ≥3 occurrences. Dotted line indicates the arithmetic mean.

FIGS. 4A-4B. Barcoding allows detection of clone cross-contamination. (FIG. 4A) Detection of two distinct barcoded clones via amplicon deep sequencing. (FIG. 4B) 3, 5, and 17 distinct barcodes were mixed and detected by amplicon deep sequencing. Dashed lines indicate the minimum read count cut-off to discriminate erroneous barcodes from genuine barcodes using an unbiased knee point detection algorithm.

### EXAMPLES

### Example 1: Targeted Integration of Individually-Labelled Polynucleotides of Interest

### 1.1 Materials and Methods

### Cell Culture and Production

All cell lines were created using a previously-generated CHO Host Cell Line containing targeted integration sites (described in WO 2019/126634 A2, which is hereby incorporated by reference in its entirety). The cells comprise single-copy landing pads providing for integration of a polynucleotide of interest via recombinase-mediated cassette exchange (RMCE).

Cells of the targeted integration CHO cell line (described in WO 2019/126634 A2) were cultured in a proprietary cell culture medium in 125-500 mL shake flask vessels at 150 rpm, 37 °C, 80% rH, and 5% CO2. Cells were passaged at a seeding density of 3-6×105 cells/mL every 3-4 days. Pools of cells stably expressing bispecific antibody molecules were generated as described in Carver, J., et al. (2020) Biotechnol Prog, 36, e2967. Briefly, expression plasmids were transfected into CHO cells by MaxCyte STX electroporation (MaxCyte, Inc). Transfected cells were then selected and expression of mAb was confirmed by flow cytometry via human IgG staining (BD FACS Canto II flow cytometer, BD). CHO clones were selected randomly during single-cell cloning by limited dilution using a confluence threshold of 10% confluence after 5 days cultivation in 96 wells.

Fed batch production cultures were performed in ambr15 bioreactors (Sartorius AG) with proprietary chemically defined production media. Cells were seeded at 2×106 cells/mL on day 0 of the production stage after adaptation to production media during 2 passages. Cultures received proprietary feed bolus on day 3, 6, 9, and 12. Cells were cultivated for 14 days. Production in the ambr15 system were operated at set points of 37 °C, DO 40%, pH 7.2, and an agitation rate of 1300 rpm.

### Generation and Analysis of Barcoded Libraries

Constructs were generated by standard cloning procedures and restriction digest cloning of the final plasmids.

Nucleotide barcodes containing a 15 nucleotide, randomized region (N15) and 10 fixed positions were introduced into the final plasmids.

For genomic DNA, DNA of 10⁸ cells was extracted using the Blood & Cell Culture DNA Maxi Kit (Qiagen) according to manufacturer's instructions. Amplicons for deep sequencing were generated with primers flanking the barcode region, 100 ng plasmid DNA as input, and 30 cycles of amplification by PCR. For detection of cellular barcodes, 2 μg of gDNA was used as input, with 30 cycles of amplification by PCR with primers flanking the barcode and one primer located outside of the RMCE integration site (to discriminate between off- and on-target integration events). Sequencing libraries were prepared using the KAPA HyperPlus Kit (Roche) using 50-100 ng (fix 20 μl purified PCR) of amplicon DNA as input, no fragmentation step, and between 20-24 cycles of amplification of PCR (post-ligation library amplification) to reach 1 μg of total DNA library per sample. Libraries were sequenced by Genewiz using the NovaSeq 6000 platform (Illumina) with 30M paired-end 150 bp reads per sample.

Barcoded cells (10×106) sampled from the ambr15® bioreactor on day 10 were washed twice in PBS and snap-frozen in liquid nitrogen. RNA extraction, Illumina stranded TruSeq RNA library preparation, poly (A) enrichment, and sequencing (NextSeq, v2.5, high.output 1*75 bp) was performed by Microsynth AG (Belgach, Switzerland). Sequences for the transgene were included manually into the reference genome (GCF_003668045.3, PICRH1.0). Reads were aligned using the hisat2 package (version 2.2.1) and transcript abundance was calculated with featureCounts (version 2.0.1). Downstream analysis was performed using PCAtools (v2.2.0) and edgeR (v3.32.1) was used to evaluate differential expression.

To characterize the diversity of the barcode libraries, forward and reverse paired-end raw reads (2×150 bp) were trimmed for universal Illumina adapters using cutadapt (v4.1) (Marcel, M. (2011) EMBnet.journal, 17, 10-12) and subsequently merged with flash (v1.2.11) (Magoc, T. and Salzberg, S. L. (2011) Bioinformatics, 27, 2957-2963). Barcodes were extracted with detection of the flanking region (antibody molecules M1: GCTTAGCCGCTTAAT AACATCTAATGCGTA (SEQ ID NO:1), M2: CTTAGCCGCTTAAT AACTTAGCTCGCGTA (SEQ ID NO:2), M3: GCTTAGCCGOTTAAT AACCTCGCTTGCGTA (SEQ ID NO:3)) and all reads which did not match the expected barcode length of 15 were discarded. Reverse complement reads were reversed with FASTX toolkit (v0.0.14). Final barcode diversity was estimated using the Chao1 capture-recapture estimator (Chao, A. (1987) Biometrics, 43, 783-791) based on barcodes observed in replicate resampling at varying depths. Collision probability (defined as the fraction of cells at start of experiment which share a barcode due to coincidence of independent barcoding events, rather than common clonal origin) was analyzed as described by Horns, F. et al., (2023) Cell, 186, 3642-3658 e3632. For a given number of cells N, N barcodes were sampled without replacement from the observed barcode pool (with sampling probability proportional to the barcode's abundance). The fraction of the sampled barcodes that were unique within the sample was calculated, designated p, such that the collision probability was 1-p.

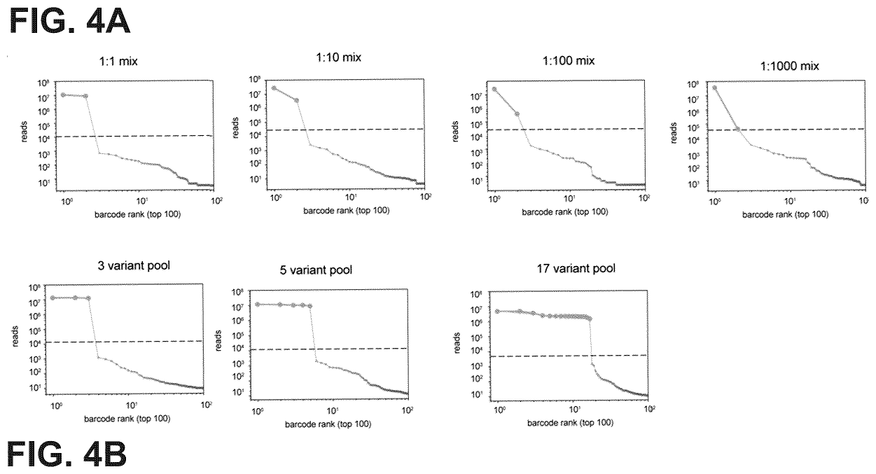

Previously characterized barcoded CHO cell lines (verified as monoclonal by fluorescent microscopy followed by barcode Sanger sequencing) were cultivated and 1×107 cells were mixed in predetermined ratios. Sequencing libraries were prepared from genomic DNA as described above. Reads were pre-processed as described above with an additional step of barcode clustering using a Levenshtein distance of 1 with Starcode (v1.4) (Zorita, E. et al., (2015) Bioinformatics, 31, 1913-1919). The number of clone barcodes was detected with an N=2 for cross-contamination, or N as indicated in FIGS. 4A-4B, using an unbiased knee point threshold based on the read count distribution (Horns, F. et al., (2023) Cell, 186, 3642-3658 e3632).

### Antibody Analytics in Supernatant

Supernatants were clarified (1000 g, 30 min, 4 °C centrifugation and 1.2 μm filtration, AcroPrep 96 Filter Plates, Pall Cooperation). Analytical protein A chromatography was performed by UHPLC with UV detection (Dionex Ultimate 3000 UHPLC fitted with POROS™ A 20 μm Column, Thermo Fisher Scientific Inc.).

Antibody integrity was analyzed after protein A affinity chromatography (PreDictor RoboColumn MabSelect SuRe, Cytiva) and normalization with protein quantitation using UV measurement (Nanoquant Infinite M200, Tecan). Percentage of correctly assembled antibodies (Main-Peak) was assessed by CE-SDS (HT Antibody Analysis 200 assay on the LabChip GXII system, PerkinElmer) under non-reducing conditions by relative quantification of the expected protein size to total protein content.

### 1.2 Single-Copy Targeted Barcode Integration can Track Diversity in Producer Cells

Most barcode delivery methods lead to non-homogeneous labeling of the population with possibly no or multiple barcode deliveries in each individual cell. In contrast, the aim of the present study was to achieve exhaustive single-copy labeling of the complete population at the time point of transfection. This was implemented within a relevant CHO system that is applicable for therapeutic protein production. Plasmid barcoding was combined with a single-copy targeted-integration recombinase-mediated cassette exchange (RMCE) strategy (Ng, D. et al., (2021) Biotechnol Progr, 37).

Pairs of plasmids (Front and Back) containing nucleotide sequences encoding antibody chains were integrated via recombinase-mediated-cassette-exchange (RMCE) into a CHO host cell line containing a landing pad (with lox acceptor sites: L3, LoxFas, L2). The Back plasmid contained a barcode library (N15), which was placed adjacent to the L2 lox site to discriminate on- and off-target integration events.

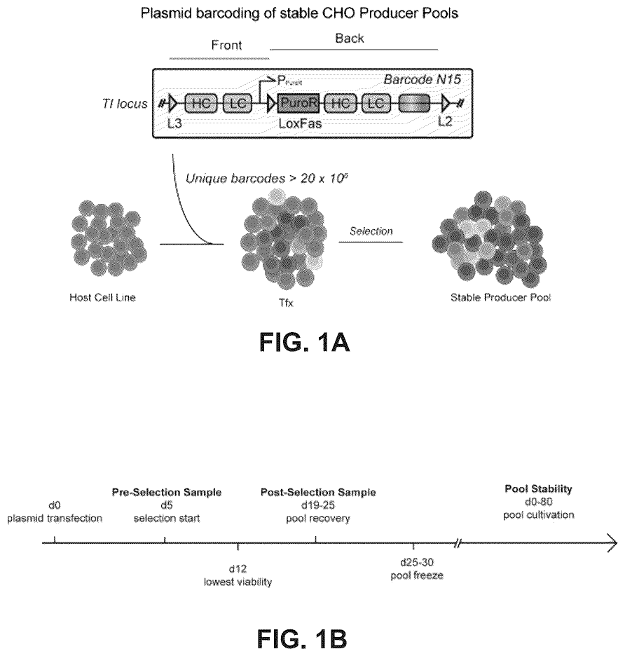

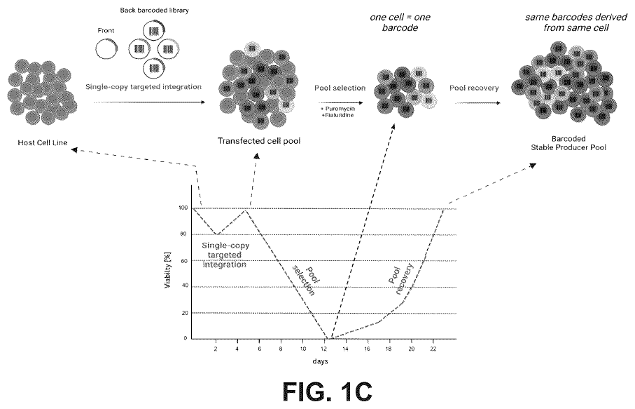

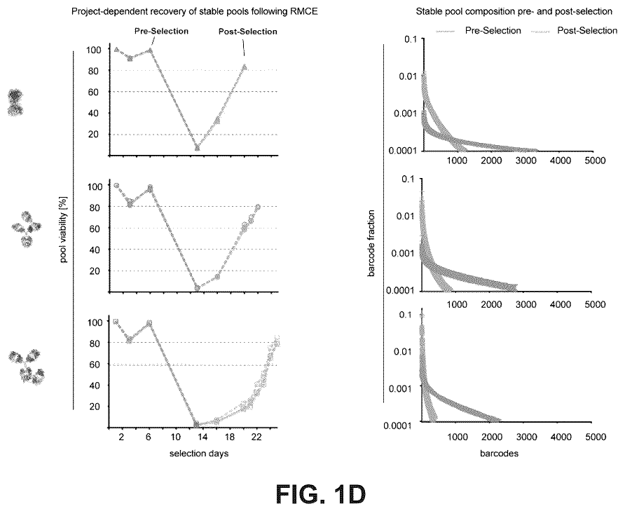

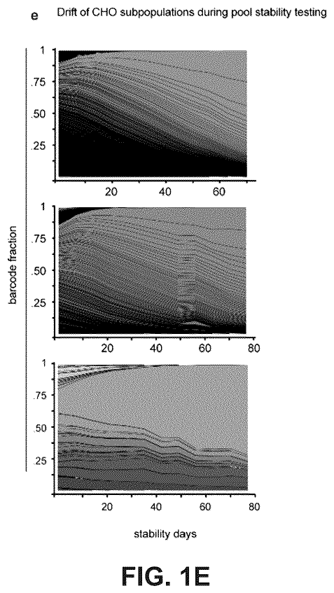

The N15 region is placed in close proximity to the genomic area outside the landing pad, allowing discrimination between on-target and off-target integration events by positioning of the primer binding sites during amplicon deep sequencing. A schematic of the targeted integration locus (post integration) is shown in FIG. 1A.

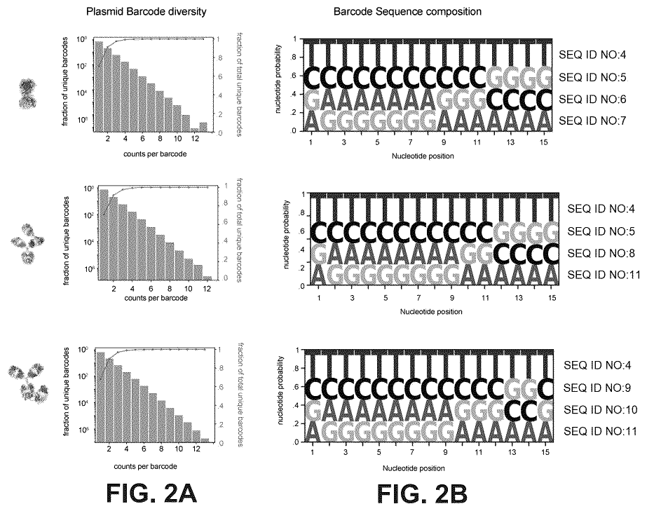

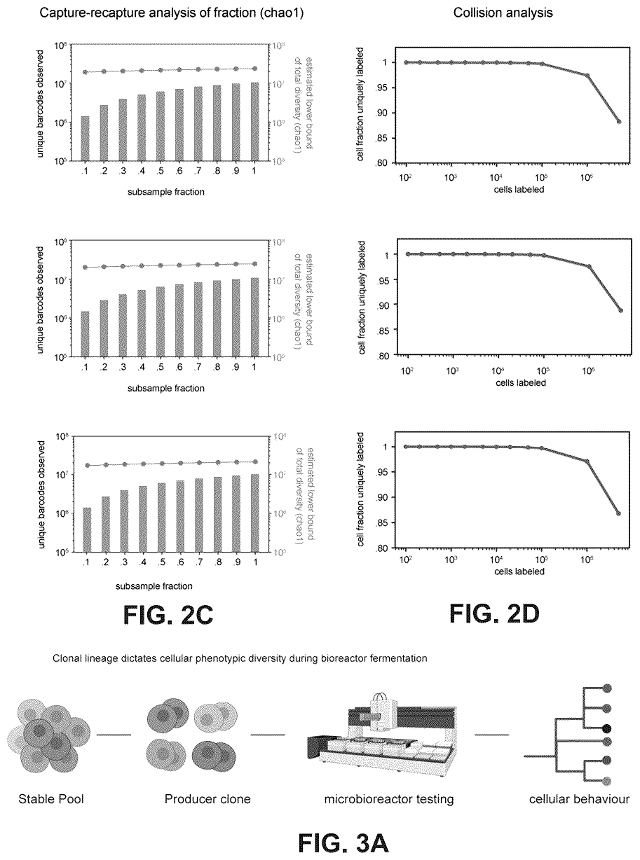

The plasmid library was validated by amplicon deep sequencing and showed a near uniform barcode representation with homogenous nucleotide composition at each position (FIGS. 2A and 2B). This provides a minimum diversity of ≥2×107, enough to label 105 cells with <0.3% collision probability (FIGS. 2C and 2D).

In stable CHO pool selections it was previously observed that the speed of CHO pool recovery, i.e. the viability loss of the cell population, is linked to the molecule encoded in the expression plasmids. To investigate a representative repertoire of therapeutic proteins produced in CHO cell lines, we selected three different antibody molecules of increasing complexity (e.g. larger plasmids, larger antibodies or greater chance of misfolding), based on the observed viability loss of the cell population during stable pool selection: 5-10%: M1 DutaFab (described e.g. in Beckmann et al. Nature Comm. (2021) 12 (708)), 1-5%: M2 TCB (T-cell bispecific antibody; described e.g. in Bacac et al., Clin Cancer Res. (2016) 22 (13): 3286-3297), <1%: M3 BS-Fusion (brain shuttle bispecific antibody; described e.g. in Weber et al., Cell Rep. (2018) 22 (1): 149-162).

Notably, the start codon of the puromycin resistance gene was placed on the Front expression vector, ensuring that only cells with in-frame and adjacent integration survive. Additionally, all cells with off-target integration of the expression plasmids do not lose the Thymidine kinase encoded in the landing pad of the Host cell line. Thus, only cells undergoing correct on-target recombination between the three LoxP sites become resistant to puromycin and survive in the presence of FIAU simultaneously. This stringent selection process ensures that all surviving cells carry a single barcode plasmid copy.

CHO host cells were transfected at day 0 with Front and Back plasmid encoding the molecule of interest and containing the barcode library. Pool composition was analyzed at two time points during stable pool selection: pre-selection at day 5 post transfection and post-selection, when the cell population reached 80% viability. Selection started at day 6 and continued until cell pool recovery (cell viability >80%). All cells reached the lowest pool viability at d13 but cell pool recovery depended on the molecule encoded on the expression plasmids. A timeline of the cell line development and evaluation process is shown in FIG. 1B, with a schematic of the cell pool selection and recovery process in FIG. 1C.

FIG. 1D shows the cell pool recovery kinetics (left panels) and the fraction of unique barcodes pre- and post-selection (right panels) for molecules M1 (top panels), M2 (middle panels), and M3 (bottom panels). The more complex the antibody being expressed, the longer the cell pool recovery period. Pool composition was approximately 3× higher at the pre-selection time point as compared to post-selection across the three molecules, indicative of rapid clone loss during the stringent selection process. Recovered stable pools consisted of a low total amount of barcodes (M1: Ø 2848, M2: Ø 1692, M3: Ø 1158), with a skewed population distribution already at post-selection. Notably, in M3 the most abundant barcode encompassed 10% of the population at the post-selection time point.

The barcodes reflect the amount of successful RMCE events and thus the pool diversity. All cell populations showed reduced numbers of barcodes after the selection process: 80-90% of successfully integrated barcodes were lost during pool selection for all three antibody molecules. Antibody complexity reduced the barcode fraction even further. At the end of selection, 95% of the M1 pool contained 2075 barcodes, 95% of the M2 pool contained 335 barcodes, while 95% of the M3 pool contained only 53 barcodes.

Next, population dynamics were analyzed in stable expression pools for a total of 11 weeks. FIG. 1E shows that pool composition drifts during prolonged cultivation and diversity decreases substantially within 80 days: in all three replicates the number of barcodes detected in each population decreased substantially with loss of 80-87% of barcode variants over the observed time course. This means that 80-90% of initial biodiversity was lost by pool splitting during routine culture. Notably, the effect is more pronounced in cases where the initial pool diversity is lower. After 12 weeks of culturing CHO cells expressing M3, the number of barcodes in the stable pool had reduced from 53 to only 7. This indicates that stable CHO pools display rapid clonal dynamics under standard passaging conditions.

Overall, these experiments demonstrate that CHO producer pools generated by RMCE display a low diversity, which further depends on the expressed molecules, and undergo rapid population skewing towards dominant clones.

### 1.3 Cells Originating from One RMCE Event Share their Production Profile and can be Distinguished from Cells Originating from Other RMCE Events

Despite sharing the genetic context and copy-number, it was previously observed that CHO cell clones generated from RMCE stable expression pools display a wide array of production relevant markers such as volumetric titer, metabolite profile and growth rates. The described genetic barcoding method allows tracing of CHO lineages from the time point of transfection onwards. This allows discrimination between related cell-clones originating from the same RMCE event (sharing the same barcode sequence) and those from different RMCE events (different barcodes).

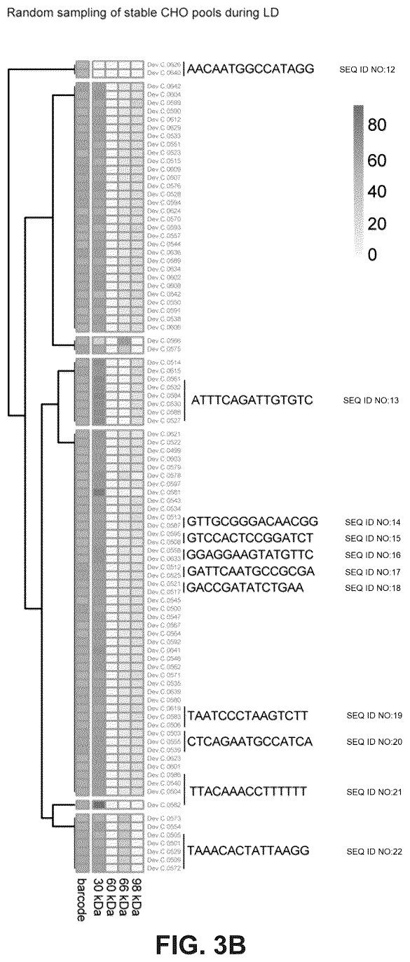

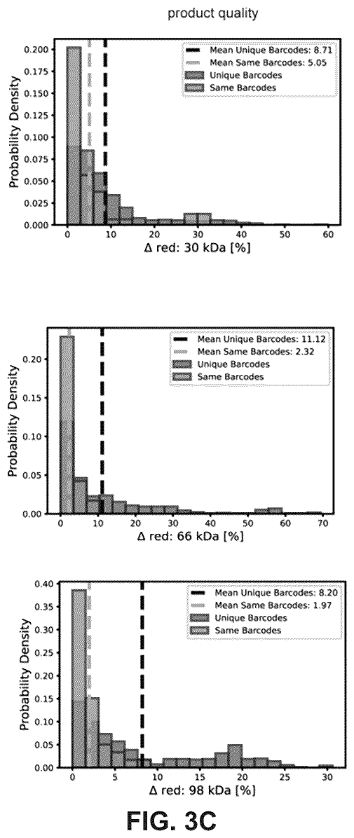

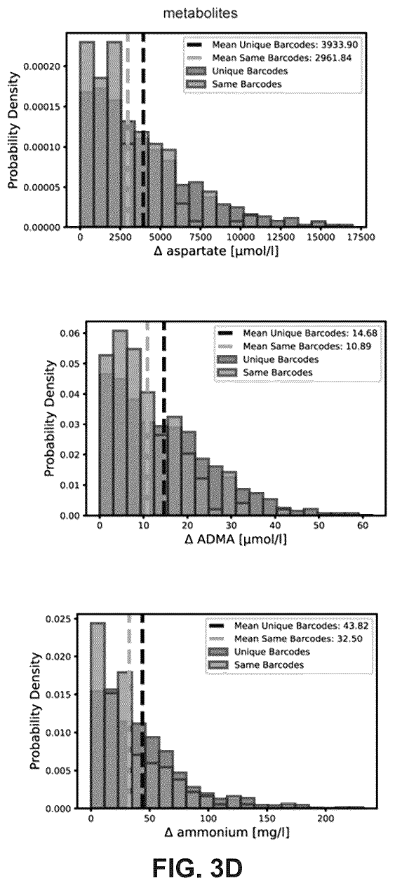

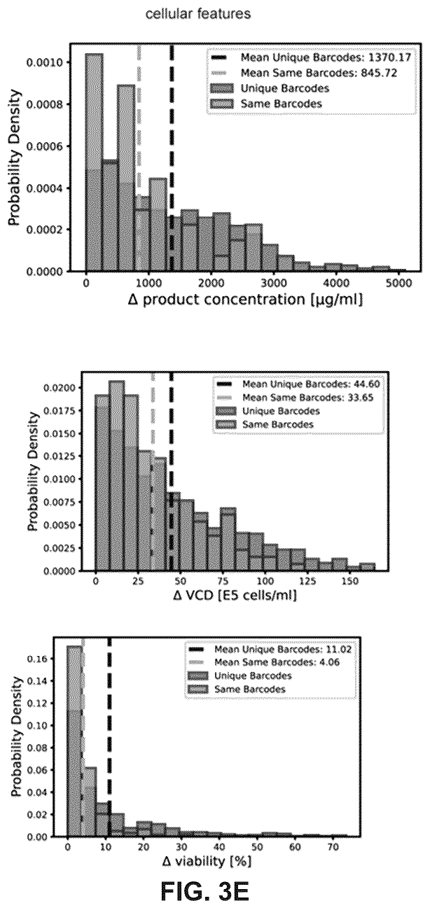

Cell-clones were generated from the barcoded CHO producer pools to test whether the observed diversity with respect to production-relevant markers lies within pre-existing cell-intrinsic factors, or if the populations underwent clonal variation during the cell line development process. Cell-clones were selected randomly during limited dilution with a confluence threshold of 10% at day 12 in the 96-well plate. The composition of barcodes within all generated clones reflected the population composition within the originating cell pool. Cell-clones were tested for production-relevant markers using a microbioreactor system, shown schematically in FIG. 3A.

Expression of individual antibody chains was analyzed after micropurification (porosA) by CE-SDS. Clustering of individual producer clones was analyzed using the binary distance matrix and complete agglomeration method. Note the distance of cells which share the same barcode.

FIG. 3B shows that cell-clones sharing the same barcode unexpectedly clustered partially based on antibody chain expression (reduced CE-SDS of purified supernatant). To holistically compare phenotypic distance between clones, pairs of absolute differences were compared within all measured bioreactor data points. Cell-clones were divided into two groups: unique barcodes with cell-clones from individual RMCE events, and same barcodes with (≥3) cell-clones from a shared RMCE event.

FIGS. 3C-3E show that most of the production-relevant parameters showed a significantly lower variance in the group of same barcodes. Notably, the relationship of product quality attributes was stronger as compared to product concentration.

This indicates that phenotypic diversity can be enriched or deceased by selecting more unique/more identical barcodes, respectively, during the cell-clone selection process. Further, the experiments demonstrate that despite some diversity remaining within cells sharing the same barcode (possibly generated by the cell line development process or inherent methodical variance), the majority of observed phenotypic diversity is likely pre-existing and cell-intrinsic.

### 1.4 Proof-of-Monoclonality by Genetic Barcoding

To limit heterogeneity of cell banks and ensure consistent product quality, proof of monoclonality has become a focus for regulatory authorities during the IND review process. While simple methods exist, such as cell sorting (flow cytometry), cell printing, or microscopic images, they are not able detect clone cross-contamination or validate the integrity of a banked cell line. Genetic barcoding offers the inclusion of a cell-intrinsic nucleotide marker which can be repetitively used to validate monoclonality and identity of a given cell line throughout the production process.

To test the sensitivity of barcode detection within this workflow, validated monoclonal cell lines were mixed together (validated by image detection and Sanger sequenced). To discriminate genuine barcodes from background introduced by sequencing errors, an unbiased knee-point filter method was included. FIG. 4A shows that the barcodes from two mixed clones were able to detect clonal cross-contamination for clone ratios of between 1:1 and 1:1000. FIG. 4B shows that 3, 5 and 17 distinct barcodes were able to be detected and differentiated.

1. A method for evaluating the diversity of a library of genetically non-identical cells having a polynucleotide of interest integrated into their genomic DNA, comprising analyzing cells obtained by:
(a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
(b) subjecting the cells obtained after step (a) to selection for cells having integrated a polynucleotide of interest into their genomic DNA;
to determine the identity of the identifier sequences integrated into their genomic DNA.

2. The method according to claim 1, wherein the method further comprises determining the proportion of cells in the population having integrated a given identifier sequence.

3. A method for obtaining a library of genetically non-identical cells having a polynucleotide of interest integrated into their genomic DNA, comprising:
(i) analyzing cells obtained by:
(a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
(b) subjecting the cells obtained after step (a) to selection for cells having integrated a polynucleotide of interest into their genomic DNA;
to determine the identity of the identifier sequences integrated into their genomic DNA; and
(ii) selecting cells determined in step (i) to have non-identical identifier sequences integrated into their genomic DNA for subsequent expansion.

4. A method for obtaining a monoclonal population of cells having a polynucleotide of interest integrated into their genomic DNA, comprising:
(i) analyzing cells obtained by:
(a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
(b) subjecting the cells obtained after step (a) to selection for cells having integrated a polynucleotide of interest into their genomic DNA;
to determine the identity of the identifier sequences integrated into their genomic DNA; and
(ii) selecting a single cell, or selecting a plurality of cells determined in step (i) to have identical identifier sequences integrated into their genomic DNA, for subsequent expansion.

5. The method of claim 1, wherein the method comprises analyzing cells obtained by:
(a) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique; and
(b) subjecting the cells obtained after step (a) to selection for cells having integrated a polynucleotide of interest into their genomic DNA;
(c) analyzing cells obtained after step (b) to determine the identity of the identifier sequences integrated into their genomic DNA; and
(d) selecting cells determined in step (c) to have non-identical identifier sequences integrated into their genomic DNA for subsequent expansion.

6. (canceled)

7. The method of claim 1, wherein the method comprises:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA; and
(iii) analyzing cells obtained after step (ii) to determine the identity of the identifier sequences integrated into their genomic DNA.

8. The method according to claim 7, wherein the method further comprises determining the proportion of cells in the population obtained after step (ii) having integrated a given identifier sequence.

9. The method of claim 3, wherein the method comprises:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting cells determined in step (iii) to have non-identical identifier sequences integrated into their genomic DNA for subsequent expansion.

10. The method of claim 4, wherein the method comprises:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion.

11. The method of claim 1, wherein the method comprises:
(i) contacting a population of cells with a plurality of vectors comprising nucleotide sequences providing for targeted integration of a polynucleotide of interest comprising an identifier sequence into the genomic DNA of a cell under conditions suitable for introducing the vectors into the cells, wherein in each vector of the plurality of vectors that comprises a polynucleotide of interest, the identifier sequence is unique;
(ii) subjecting the cells obtained after step (i) to selection for cells having integrated a polynucleotide of interest into their genomic DNA;
(iii) analyzing cells obtained after step (ii) to determine the identity of the identifier sequences integrated into their genomic DNA;
(iv) selecting a single cell, or a plurality of cells determined in step (iii) to have identical identifier sequences integrated into their genomic DNA, for subsequent expansion; and
(v) analyzing cells obtained after step (iv) to determine the identity of the identifier sequences integrated into their genomic DNA.

12. The method according to claim 11, wherein the method for evaluating the diversity of library of genetically non-identical cells having a polynucleotide of interest integrated into their genomic DNA is a method for monitoring or evaluating the monoclonality of a putative monoclonal population of cells having a polynucleotide of interest integrated into their genomic DNA.

13-22. (canceled)

23. The method of claim 1, wherein prior to integration of a polynucleotide of interest, the cells comprise genomic DNA having at least one single-copy landing pad providing for targeted integration of a polynucleotide of interest.

24. The method of claim 1, wherein the polynucleotide of interest comprises a nucleotide sequence encoding one or more polypeptides of interest.

25. The method of claim 24, wherein the one or more polypeptides of interest are each independently selected from the group consisting of: an antigen-binding polypeptide, an aptamer, a constituent polypeptide of an antigen-binding polypeptide complex, an antibody/antigen-binding fragment or derivative thereof, a constituent polypeptide of an antibody/antigen-binding fragment or derivative thereof, an Fc fusion polypeptide, an anticoagulant, a blood factor, a bone morphogenetic polypeptide, a decoy receptor for a ligand, a decoy ligand for a receptor, an enzyme, an enzyme co-factor, a growth factor, a hormone, an interferon, an interleukin, a thrombolytic, a transcription factor, an epigenetic modifier, a constituent polypeptide of a site-specific nuclease nucleic acid editing system, a constituent polypeptide of a ribonucleoprotein, a viral polypeptide, or a polypeptide useful for the production of a biomolecule.
